(19) United States
(12) Patent Application Publication
(10) Pub. No.: US 2026/0335330 A1
(43) Pub. Date: Oct. 29, 2026

(54) FUSED AMINO PYRIMIDINE COMPOUNDS FOR TREATMENT OF CANCER

(71) Applicant: Ovid Therapeutics Inc., New York, NY (US)

(72) Inventors: Jeremy Levin, New York, NY (US); Zhong Zhong, Hingham, MA (US)

(21) Appl. No.: 19/653,855

(22) Filed: Apr. 21, 2026

Related U.S. Application Data
(60) Provisional application No. 63/792,008, filed on Apr. 21, 2025.
(60) Provisional application No. 63/942,167, filed on Dec. 16, 2025.

(51) Int. Cl. A61K 31/5377; A61K 31/192; A61K 31/203; A61K 31/337; A61K 31/4184; A61K 31/4745; A61K 31/517; A61K 31/519; A61K 31/52; A61K 31/53; A61K 31/706; A61P 35/00

(52) U.S. Cl. CPC A61K 31/5377; A61K 31/192; A61K 31/203; A61K 31/337; A61K 31/4184; A61K 31/4745; A61K 31/517; A61K 31/519; A61K 31/52; A61K 31/53; A61K 31/706; A61P 35/00

(57) ABSTRACT

Methods and compositions including a compound of Formula (I), or a pharmaceutically acceptable salt thereof, are provided for use in treating cancer and inhibiting cancer proliferation and metastasis. Methods and compositions for treating cancer are provided that reduce or prevent multidrug resistance (MIDR) in cancer cells. Methods and compositions are provided for decreasing activity of breast cancer resistance protein (BCRP).

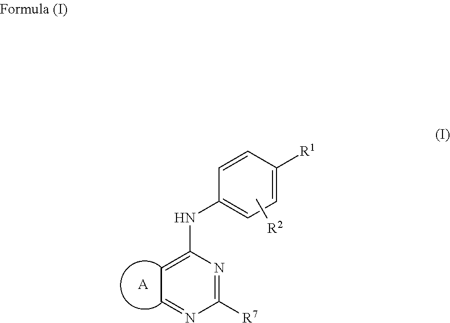

wherein R¹, R², R⁷ and ring A have any of the meanings herein defined in the description. are provided.

### CROSS REFERENCE TO RELATED APPLICATIONS

This application claims benefit of and priority to the U.S. Provisional Application No. 63/792,008, filed on Apr. 21, 2025, 2025, and the U.S. Provisional Application No. 63/942,167, filed on Dec. 16, 2025, which are incorporated herein by reference in their entirety.

### TECHNICAL FIELD

Treatment of cancer with fused amino pyrimidine compounds.

### BACKGROUND

According to the World Health Organization, (https://www.who.int/news-room/fact-sheets/detail/cancer) cancer is a generic term for a large group of diseases that can affect any part of the body. Other terms used are malignant tumors and neoplasms. Cancer is a leading cause of death worldwide, accounting for nearly 10 million deaths in 2020, or nearly one in six deaths. A defining feature of cancer is the rapid creation of abnormal cells that grow beyond their usual boundaries, and which can then invade adjoining parts of the body and spread to other organs; the latter process is referred to as metastasis. Widespread metastases are the primary cause of death from cancer.

Medulloblastoma (MB) is the most common malignant pediatric brain tumor. According to OMIM #155255, MB accounts for 16% of all pediatric brain tumors, and 40% of all cerebellar tumors in childhood are MB. Approximately 10 to 15% of medulloblastomas are diagnosed in infancy. MB occurs bimodally, with peak incidences between 3 and 4 years and 8 and 9 years of age. MB accounts for less than 1% of central nervous system (CNS) tumors in adults, with highest incidence in adults 20 to 34 years of age.

MB is defined by four molecular subgroups (Wnt, Shh, Group 3, Group 4) based on transcriptional and epigenetic profiles. See, Manoranjan, et al. Nat Commun 11, 4323 (2020). The Wnt (wingless) and Shh (Sonic Hedgehog) MBs are named for the signaling pathways thought to play prominent roles in the pathogenesis of that subgroup. The Wnt signaling pathways are a group of signal transduction pathways which begin with proteins that pass signals into a cell through cell surface receptors. NT-activated medulloblastomas are defined by activating mutations in the WNT/β-catenin signaling pathway (e.g., CTNNB1 or germline APC), are often associated with loss of chromosome 6, and have the most favorable prognosis. Manoranjan, et al., supra. Wnt MB accounts for 10% of cases. Id.

The Shh signaling pathway is one of the major trafficking networks that regulates the key events during developmental processes, i.e., growth and patterning of multicellular embryos. Aberration in the regulation and transduction of the Shh signaling pathway has been implicated in birth defects, tissue regeneration, stem cell renewal and cancer growth. Choudhry et al., Ann Neurosci. 2014; 21(1):28-31. SHH-activated medulloblastomas (SHH-MB) account for 25-30% of all MBs and occur with a bimodal age distribution, encompassing many infant and adult, but fewer childhood cases. Menyhart and Gyorffy, Annals of Clinical and Translational Neurology 2019; 6(5): 990-1005. Different age groups are characterized by distinct survival outcomes and age-specific alterations of regulatory pathways. Id.

Group 3 MB has a photoreceptor/retinal expression signature, while group 4 MB expresses neuronal genes. Tamayo-Orrego, Lukas, and Frédéric Charron. 1000Research vol. 8 F1000 Faculty Rev-1823. 29 Oct. 2019. The current idea is that each different MB group is derived from a specific cell of origin, which therefore determines the clinical and molecular behavior of the disease. Id.

Glioma is a type of brain tumor that develops from the glial cells, which are specialized cells that surround and support neurons in the brain. According to the National Institutes of Health (NIH), Genetic and Rare Diseases Information Center (GARD), glioma is generally classified by the type of glia cell that is involved in the tumor: astrocytoma-tumors that develop from star shaped glial cells called astrocytes; ependymomas-tumors that arise from ependymal cells that line the ventricles of the brain and the center of the spinal cord; oligodendrogliomas-tumors that affect the oligodendrocytes, the myelinating glia of the central nervous system. Gliomas are graded on a scale of one to four. Grade one gliomas usually grow slowly and frequently behave in a more benign fashion. Grade two and grade three gliomas can grow more quickly and they frequently require more aggressive treatment. Grade four gliomas are the most aggressive type and are also known as glioblastoma. Symptoms of glioma vary by type but may include headaches, nausea and vomiting, confusion, personality changes, trouble with balance, vision problems, speech difficulties, and/or seizures. In studies, 8 members of 19 WNTs, i.e., WNT1, 2, 2B, 3A, 5A, 6, 7A and 7B, were shown as being related with glioma development. Xu et al., Bioscience reports vol. 40 (3) (2020).

Breast cancer originates in breast tissue and cells usually form a tumor which may be in situ or invasive. According to the NIH, breast cancer is the second most common type of cancer in women in the United States. Rarely, it can also affect men. Certain genetic markers are associated with the risk of breast cancer such as BRCA1, BRCA2 and HER2 which may raise the risk of ovarian and other cancers. The different types of breast cancer are typically based on which breast cells turn into cancer. The types include ductal carcinoma, which begins in the cells of the ducts and is the most common type; lobular carcinoma, which begins in the lobules and is more often found in both breasts than other types of breast cancer; inflammatory breast cancer, in which cancer cells block lymph vessels in the skin of the breast; Paget's disease of the breast, which is a cancer involving the skin of the nipple. The signs and symptoms of breast cancer include a new lump or thickening in or near the breast or in the armpit; change in the size or shape of the breast; a dimple or puckering in the skin of the breast; a nipple turned inward into the breast; nipple discharge other than breast milk; scaly, red, or swollen skin in the nipple area or the breast; and pain in any area of the breast.

Squamous cell carcinoma is the second most common form of skin cancer, characterized by abnormal, accelerated growth of cells that make up the middle and outer layers of the skin. According to the Mayo Clinic and Healthline, squamous cell carcinoma of the skin is usually not life-threatening, though it can be aggressive. Squamous cell carcinoma can also be found in other areas of the body, including the mouth and lungs, but it is much rarer. Most squamous cell carcinomas of the skin result from prolonged exposure to ultraviolet (UV) radiation. Signs and symptoms of squamous cell carcinoma of the skin include a firm, red nodule; a flat sore with a scaly crust; a new sore or raised area on an old scar or ulcer; a rough, scaly patch on the lip that may evolve to an open sore; a red sore or rough patch inside the mouth; and a red, raised patch or wartlike sore on or in the anus or genitals.

Lung cancer originates in the lungs and may spread to lymph nodes or other organs in the body. As many as 40% of people with lung cancer develop brain metastases. According to the Centers for Disease Control and Prevention (CDC), lung cancers are grouped into two main types called small cell and non-small cell (including adenocarcinoma and squamous cell carcinoma). These types of lung cancer grow differently and are treated differently. Non-small cell lung cancer is more common than small cell lung cancer. People who smoke tobacco have the greatest risk of lung cancer. Lung cancer typically doesn't cause signs and symptoms in its earliest stages. Signs and symptoms of lung cancer typically occur when the disease is advanced and may include a new cough that doesn't go away; coughing up blood; shortness of breath; chest pain; hoarseness; losing weight without trying; bone pain; and headache.

Cancer of the peritoneum is cancer that has started in or has spread to the abdominal cavity. According to the NIH, whether the cancer starts in the peritoneum or spreads from somewhere else (i.e., the ovaries, colon, appendix, etc.), it is considered advanced once it is in the peritoneum and can be referred to as peritoneal carcinomatosis. Patients in the early stages will often have few symptoms until the disease is advanced. When symptoms occur, they are often vague and nonspecific, such as fatigue; abdominal swelling; diffuse abdominal pain; frequent urination; bowel changes; abnormal vaginal bleeding; an abdominal mass; unintended weight loss; and a sense of fullness when eating. As the disease progresses, fluid may build up in the abdomen, causing additional abdominal discomfort, nausea and vomiting, and shortness of breath. Complications of peritoneal cancer may include bowel and urinary tract obstruction, sometimes requiring a stent or nephrostomy tube.

Hepatocellular cancer is the most common type of primary liver cancer. According to the Mayo Clinic and the American Cancer Society, hepatocellular carcinoma occurs most often in people with chronic liver diseases, such as cirrhosis caused by hepatitis B or hepatitis C infection, as well as people who drink large amounts of alcohol and who have an accumulation of fat in the liver. Most patients do not demonstrate symptoms in the early stages of primary liver cancer. When symptoms do appear, they may include losing weight without trying; loss of appetite; upper abdominal pain; nausea and vomiting; general weakness and fatigue; abdominal swelling; jaundice; and white, chalky stools. GABA can be a by-product of liver disease and contributes to hepatic encephalopathy in patients with cirrhosis, which can further lead to hepatocellular cancer. Decreases in GABAergic activity may contribute to the pathogenesis of hepatocellular carcinoma.

Gastric cancer can affect any part of the stomach. In most of the world, stomach cancers form in the stomach body. However, according to the Mayo Clinic, in the United States, stomach cancer is more likely to affect the gastroesophageal junction. Although it is not clear what causes gastric cancer, factors that increase the risk include gastroesophageal reflux disease; obesity; a diet high in salty and smoked foods; a diet low in fruits and vegetables; family history; infection with Helicobacter pylori; gastritis; smoking; and stomach polyps. Symptoms of stomach cancer may include difficulty swallowing; feeling bloated after eating; feeling full after eating small amounts; heartburn; indigestion; nausea; stomach pain; unintentional weight loss; and vomiting. GABA has been linked to the proliferation of gastrointestinal cancer types in several studies and has served as a tumor signaling molecule for gastric cancer.

Pancreatic cancer can develop from two kinds of cells in the pancreas: exocrine cells and neuroendocrine cells, such as islet cells. According to the National Cancer Institute, the exocrine type is more common and is usually found at an advanced stage. Pancreatic neuroendocrine tumors, also called islet cell tumors, are less common but have a better prognosis. It's not clear what causes pancreatic cancer, but some factors that may increase the risk include smoking and having inherited gene mutations. Symptoms of pancreatic cancer often do not occur until the disease is advanced and may include abdominal pain that radiates to the back; loss of appetite or unintended weight loss; jaundice; light-colored stools; dark-colored urine; itchy skin; new diagnosis of diabetes or existing diabetes becoming more difficult to control; blood clots; and fatigue. GABA has been linked to stimulation of pancreatic cancer growth.

Glioblastoma, also referred to as a grade IV astrocytoma, is a fast-growing and aggressive brain tumor. It invades the nearby brain tissue, but generally does not spread to distant organs. According to the American Association of Neurological Surgeons, glioblastomas can arise in the brain de novo or evolve from lower-grade astrocytoma. The cause of glioblastomas is unknown, but glioblastomas tend to occur more often in older adults and men. Symptoms may vary based on the location of the tumor but may include persistent headaches; double or blurred vision; vomiting; loss of appetite; changes in mood and personality; changes in ability to think and learn; new onset of seizures; gradual speech difficulty.

Cervical cancer occurs in the lower part of the uterus that connects to the vagina. According to the CDC, various strains of the human papillomavirus, a sexually transmitted infection, play a role in causing most cervical cancer. In a small percentage of people, the virus can survive for years, contributing to the process that causes some cervical cells to become cancer cells. Early-stage cervical cancer generally produces no symptoms. Symptoms of more-advanced cervical cancer include vaginal bleeding after intercourse, between periods, or after menopause; watery, bloody vaginal discharge that may be heavy and have a foul odor; and pelvic pain or pain during intercourse.

Ovarian cancer originates in the ovaries, or in the related areas of the fallopian tubes or the peritoneum. According to the CDC, ovarian cancers exhibit a variety of different tumor types. The most common tumor type is high-grade serous carcinoma, occurring in about 70% of ovarian cancer cases. The cause of ovarian cancer is unknown, but risk factors include family history, genetic mutation of BRCA1 or BRCA 2, endometriosis, and being middle aged or older. Signs and symptoms of ovarian cancer may include abdominal bloating or swelling; weight loss; discomfort in the pelvic area; fatigue; back pain; changes in bowel habits; and frequent urination. Ovarian cancer has been associated with GABA and it may be used for detection.

Bladder cancer most often begins in the urothelial cells that line the bladder, ureters, and kidneys. According to the American Cancer Society, older adults are at a higher risk for bladder cancer. Most bladder cancers are diagnosed at an early stage when the cancer is highly treatable, but bladder cancer has a high reoccurrence rate. Symptoms of bladder cancer include blood in the urine; frequent urination; painful urination; and back pain. GABA has been associated with the promotion and detection of bladder cancer in several studies.

Colorectal cancer originates in the colon or rectum and can be called either colon cancer or rectal cancer, depending on the source. According to the American Cancer Society, most colorectal cancers start as a polyp on the inner lining of the colon or rectum. Not all polyps become cancerous, but depending on the type of polyp it is, the risk can be higher or lower. Hyperplastic polyps and inflammatory polyps are very low risk. Adenomatous polyps, sessile serrated polyps, and traditional serrated adenomas are high risk and referred to as “pre-cancerous.” Symptoms of colon cancer include a persistent change in bowel habits, including diarrhea or constipation, or a change in the consistency of the stool; rectal bleeding or blood in stool; persistent abdominal discomfort, such as cramps, gas or pain; weakness or fatigue; and unexplained weight loss. GABA has been associated with the proliferation and detection of colorectal cancer in several NIH sponsored studies.

Endometrial or uterine carcinoma begins in the lining or glands of the uterus. Most cases occur in women over the age of 55. Risk factors for endometrial or uterine cancer include obesity, family history, age, and hormone effectors (e.g., estrogen, birth control, pregnancy, and polycystic ovarian syndrome). According to the Mayo Clinic, a key symptom is abnormal vaginal bleeding, such as bleeding after menopause or bleeding between periods. Other symptoms may include pelvic pain and pain during sex, but some women experience no symptoms at all. Endometrial and uterine cancers have been associated with GABA and have GABA receptors that facilitate cancer proliferation and can be used for detection of abnormalities.

Salivary gland cancer begins in one of the salivary glands, including the parotid glands, submandibular glands, and sublingual glands. According to the Mayo Clinic, most salivary gland tumors occur in the parotid glands. Most salivary gland tumors are benign, but sometimes they can be cancerous. Symptoms of a salivary gland tumor may include a lump or swelling on or near the jaw or in the neck or mouth; numbness in the face; muscle weakness on one side of the face; persistent pain in the area of a salivary gland; difficulty swallowing; and trouble opening the mouth widely.

Kidney/renal cancer begins in the lining of tubules in the kidney. According to the Mayo Clinic, the incidence of kidney cancer seems to be increasing, which is most likely due to technological improvements in testing and imaging. Risk factors for renal cancer include smoking, abusing pain medications, obesity, family history, and high blood pressure. Kidney cancer can occur at any age, but young children are more likely to develop a kind of kidney cancer called Wilms' tumor. Kidney cancer is often discovered at an early stage, when the cancer is small, confined to the kidney, and usually does not have symptoms. Later stage symptoms may develop, including blood in urine; pain in back or side that persists; loss of appetite; unexplained weight loss; tiredness; and fever. GABA has emerged as a tumor signaling molecule that controls proliferation in renal cancers and can be used for detecting its presence.

Prostate cancer occurs in the male prostate and is the most common cancer among men in the United States. According to the American Cancer Society, prostate cancer usually grows very slowly and finding and treating it before symptoms occur may not improve the patient's health or help them live longer. Prostate cancer is rare in men under 40, but the risk of developing prostate cancer rises rapidly after age 50. Additionally, inherited mutations of the BRCA1 or BRCA2 genes can increase risk. Prostate cancer may cause no signs or symptoms in its early stages, but more advanced stages may cause symptoms such as: trouble urinating; decreased force in the stream of urine; blood in the urine; blood in the semen; bone pain; losing weight without trying; and erectile dysfunction. GABA has been reported to increase cellular proliferation associated with prostate cancers.

Vulvar cancer is a rare cancer that occurs on the outer surface of female genitalia, including the labia and clitoris. Though it can occur at any age, it is most common in older adults. According to the NIH, persistent infection of the human papillomavirus can increase risk of vulvar cancer, as may smoking. Symptoms of vulvar cancer may include persistent itching; localized pain and tenderness; bleeding that is not from menstruation; skin changes, such as color changes or thickening; and lumps, wartlike bumps, or open sores.

Thyroid cancer originates in the thyroid gland. The two most common types of thyroid cancers are papillary and follicular. Papillary thyroid cancer is the most common type and can occur at any age. Papillary thyroid cancer tends to grow slowly and often spreads to lymph nodes in the neck. Follicular cancer is less common and can spread through the blood to distant organs, particularly the lungs and bones. According to the American Thyroid Association, factors that may increase risk for thyroid cancer include history of exposure to high doses of radiation, family history, and being above the age of 40. Most thyroid cancers do not cause any symptoms in early stages, but as thyroid cancer grows, it may cause a nodule that can be felt through the skin on the neck; changes of the voice, including increasing hoarseness; difficulty swallowing; swollen lymph nodes in the neck pain in the neck and throat. GABA alterations have also demonstrated effects on proliferation of thyroid cancers.

Head and neck cancers usually begin in the squamous cells that line the mucosal surfaces of the head and neck, such as inside the oral cavity, throat, and the larynx. Less common head and neck cancers include paranasal sinus and nasal cavity cancers as well as salivary gland cancers. Alcohol and tobacco are major risk factors for cancers of the head and neck. Cancers of the oropharynx are also strongly linked to human papillomavirus. UV light can contribute to risk of cancer on the lips. An infection with the Epstein-Barr virus can raise the risk of cancers in the nose, behind the nose, and cancers of the salivary glands. Chemical and radiation exposures, including wood dust, formaldehyde, asbestos, nickel, etc., can increase the risk of nasopharynx cancers. Symptoms can vary based on the type of cancer but can include open sores that persist; unusual pain or bleeding; trouble with chewing, swallowing, and speaking; and localized swelling. GABA has demonstrated a proliferating effect on some head and neck cancers, including head and neck squamous cell carcinoma.

B-Cell lymphoma is a type of non-Hodgkin lymphoma that originates in the B-cells. There are many subtypes that fall in this category. According to the American Cancer Society, diffuse large B-cell lymphoma (DLBCL) and follicular lymphoma the most common subtypes. DLBCL is the most common type and occurs mostly in older adults. DLBCL tends to be aggressive and fast-growing. Follicular lymphoma is less common and less aggressive and is very rare in young people. Symptoms of non-Hodgkin's lymphomas may include swollen lymph nodes in the neck, armpits or groin; abdominal pain or swelling; chest pain, coughing or trouble breathing; persistent fatigue; fever; night sweats; and unexplained weight loss.

Chronic lymphocytic leukemia (CLL) is another subtype of B-Cell non-Hodgkin lymphoma where most of the cancer cells are found in the blood and bone marrow. CLL progresses slowly and usually affects older adults. The exact mechanism that causes CLL is unknown, but age and chemical exposures appear to be risk factors. According to the American Cancer Society, CLL may not cause any symptoms in the early stages, but when symptoms do occur, they may include swollen lymph nodes; fatigue; fever; pain due to an enlarged spleen; night sweats; weight loss; frequent infections; and easy bruising.

Acute lymphoblastic leukemia (ALL) is a type of cancer of the blood and bone marrow that affects white blood cells. According to the American Cancer Society, ALL is the most common childhood cancer, and it is very aggressive and fast acting. Possible risk factors for ALL include exposure to x-rays before birth; exposure to radiation; certain changes in chromosomes or genes; and certain genetic conditions, such as down syndrome; neurofibromatosis type 1; bloom syndrome; and constitutional mismatch repair deficiency. Symptoms may include enlarged lymph nodes; bruising; fever; bone and joint pain; bleeding from the gums; shortness of breath; fatigue and weakness; loss of appetite; and frequent infections. GABA is involved in the proliferation and spread of ALL and has been observed in several studies.

Hairy cell leukemia (HCL) is another, very rare, subtype of B-Cell non-Hodgkin lymphoma. The affected cells are B lymphocytes found in the bone marrow, spleen, and blood with projections that give a “hairy” appearance. According to the Mayo Clinic, HCL affects more men than women, and it occurs most commonly in middle-aged or older adults. HCL is considered a chronic disease because it may never completely disappear, although treatment can lead to a remission for years. Some people have no signs or symptoms of hairy cell leukemia, but a blood test for another disease or condition may inadvertently reveal hairy cell leukemia. When symptoms do occur, they may include a feeling of fullness in the abdomen that makes it uncomfortable to eat more than a little at a time; fatigue; easy bruising; recurring infections; weakness; and unintentional weight loss.

Chronic myeloid leukemia (CML) is an uncommon type of cancer that originates in certain blood-forming cells of the bone marrow. CML is a slow growing leukemia, but it can change into a fast-growing acute leukemia that is difficult to treat. CML occurs mostly in adults, but very rarely it occurs in children, too. According to the Mayo Clinic, CML often does not cause symptoms and might be detected during a blood test. When symptoms occur, they may include bone pain; easy bleeding; feeling full after eating a small amount of food; fatigue; fever; unintentional weight loss; loss of appetite; pain or fullness below the ribs on the left side; and night sweats.

Acute myeloid leukemia (AML) is a cancer of the blood and bone marrow. It is the most common type of acute leukemia in adults and is very aggressive and progresses rapidly. According to the Mayo Clinic, males and adults over the age of 65 are at higher risk of AML, as well as smokers, those with prior exposure to radiation and dangerous chemicals, and those with other blood disorders. Symptoms of early-stage AML may mimic those of the flu or other common diseases. Later stages may have symptoms including fever; bone pain; lethargy and fatigue; shortness of breath; pale skin; frequent infections; easy bruising; and unusual bleeding, such as frequent nosebleeds and bleeding from the gums.

Melanoma is the most dangerous type of skin cancer. It grows quickly and can spread to any organ. Melanoma originates in melanocytes which produce melanin. According to National Cancer Institute, the exact cause of melanomas is not clear, but exposure to ultraviolet radiation from sunlight or tanning lamps/beds increases the risk of developing melanoma. The risk of melanoma seems to be increasing in people under 40, especially women. The first melanoma symptoms often are a change in an existing mole or the development of a new pigmented or unusual-looking growth on the skin. Malignant moles vary greatly in appearance. Some signs that a pigmentation may be malignant are if the growth is asymmetrical in shape; has an irregular border; changes in color; has a growth in diameter; or appears to be evolving in color or shape. Melanomas and GABA have demonstrated a direct relationship and melanomas have GABA receptors that proliferate cancerous growth.

Testicular cancer occurs in the testes and is the most common cancer in American males between ages 15 and 35. There are two primary forms: seminoma and non-seminoma. Seminoma is slow-growing and primarily affects people in ages 40-60. Non-seminoma grows more rapidly and affects mainly people in their teens, 20s, and 30s. Symptoms of testicular cancer include a lump or enlargement in either testicle; a feeling of heaviness in the scrotum; a dull ache in the abdomen or groin; a sudden collection of fluid in the scrotum; pain or discomfort in a testicle or the scrotum; enlargement or tenderness of the breasts; and back pain. Testicular cancer usually affects only one testicle. Testicular cancer and GABA have demonstrated a direct relationship and wherein GABA proliferates cancerous cell growth and sperm motility changes.

Brain metastases (metastatic brain tumors) and leptomeningeal metastasis happen when cancer spreads from an origination or primary cancer site. Brain metastases occur when cancer cells travel to the brain itself. Leptomeningeal metastasis (also called leptomeningeal cancer, leptomeningeal carcinomatosis, leptomeningeal disease (LMD), neoplastic meningitis, meningeal metastasis and meningeal carcinomatosis) is a rare complication of cancer in which the disease spreads from the original tumor site to the meninges surrounding the brain and spinal cord. Leptomeninges are the two innermost layers of tissue that cover the brain and spinal cord. Leptomeningeal metastasis occurs when the cancer cells invade the cerebrospinal fluid and spread throughout the central nervous system. The prognosis is generally poor with survival typically measured in months. The most common cancers to include the leptomeninges are breast cancer, lung cancer, and melanomas because they can metastasize to the subarachnoid space. The condition can also arise from primary brain tumor like medulloblastoma or glioma. The most common symptoms of leptomeningeal cancer are pain and seizures. The other symptoms may include headaches (usually associated with nausea, vomiting, light-headedness), gait difficulties from weakness or ataxia, memory problems, incontinence, sensory abnormalities. In some cases, symptoms may include double vision, numb chin, back pain, leg weakness, sphincter-related problems, hydrocephalus, loss of urine control, difficulty walking, limb weakness and paresthesia, bowel and bladder dysfunction, double vision, trigeminal sensory or motor loss, cochlear dysfunction, confusion and cognitive impairment.

Many cancers are treated with surgery, chemotherapy, radiation, or combinations thereof. Chemotherapeutic agents used in the treatment of cancer are known to produce serious and unpleasant side effects in patients. For example, some chemotherapeutic agents can cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhea, decreased immunity, anemia, high tone hearing loss, cardiotoxicity, fatigue, neuropathy, myelosuppression, to name a few. In some instances, chemotherapy is not effective or loses effectiveness after a period of efficacy, either during treatment, or shortly after the treatment regimen concludes (i.e., the treatment regimen does not result in a cure).

Indeed, cancer cells frequently develop resistance to structurally and mechanistically unrelated drugs over a period of time. This phenomenon of resistance is known as multidrug resistance (MDR). Tumor cells can develop mechanisms to actively pump out chemotherapy drugs (efflux), reducing their intracellular concentration and effectiveness. MDR studies suggested that drug transport is a carefully controlled process, and this process was later found to be regulated by members of the ATP-binding cassette (ABC) transporter family of proteins. See, Sun Y L, Patel A, Kumar P, Chen Z S; Role of ABC transporters in cancer chemotherapy; Chin J Cancer. 2012 February; 31(2):51-57. The overexpression of specific ABC transporters in cancer cell lines and tumors produces MDR, which is the major factor contributing to the failure of chemotherapy. Id. Three ABC transporters appear to account for most of the reported MDR in humans: P-gp, multidrug resistance protein 1 (MRP1) or ABCC1, and breast cancer resistance protein (BCRP) also known as ABCG2 or MXR or ABCP. Id. Breast cancer resistance protein was so-named because it was isolated from multidrug-resistant human breast cancer cells. The high expression of BCRP in breast cancer, colon cancer, small cell lung cancer, ovarian cancer, and melanoma is the main cause of MDR in these and other tumors.

Improved methods for the treatment of cancer, and compositions capable of delivering bioactive agents to aid in the treatment of cancer remain desirable. Moreover, in view of the high levels of mortality attributable to cancer, there exists a need for additional treatments to combat the disease.

Fused amino pyridine compounds and pharmaceutically acceptable salts thereof are disclosed in WO2021/180952 (US Publication No. 20230151013A1), incorporated herein by reference.

### SUMMARY

Methods and compositions for treating cancer are provided. In embodiments, methods for treating cancer include administering an effective amount of a compound according to Formula (I):

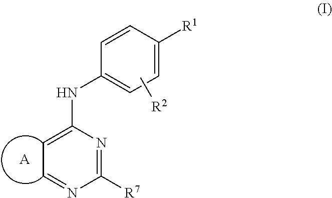

or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from

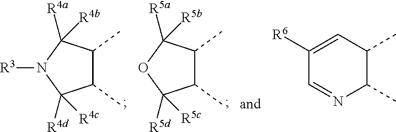

or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to a subject in need thereof.

In embodiments, compositions for treating cancer which include a compound according to Formula (I), or a pharmaceutically acceptable salt thereof, are administered to a subject in need thereof. A subject in need thereof may be a subject that has been diagnosed with cancer. In embodiments, the cancer is medulloblastoma, glioma, breast cancer, squamous cell cancer, melanoma, lung cancer, small cell lung cancer, cancer of the peritoneum, hepatocellular cancer, gastric cancer, colon cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia (CLL), acute lymphoblastic leukemia (ALL), hairy cell leukemia, leptomeningeal carcinomatosis, or chronic myeloblastic leukemia and subtypes thereof. In embodiments, an effective amount of a compound according to Formula (I), or a pharmaceutically acceptable salt thereof, is administered. In embodiments, a compound according to Formula (I), or a pharmaceutically acceptable salt thereof, inhibits activity of breast cancer resistance protein (BCRP). In embodiments, methods of inhibiting BCRP or decreasing the activity of BCRP, include contacting cells expressing BCRP with a compound according to Formula (I), or a pharmaceutically acceptable salt thereof.

In embodiments, a method of decreasing activity of breast cancer resistance protein (BCRP) is provided which includes contacting cells expressing BCRP with a compound according to Formula (I), or a pharmaceutically acceptable salt thereof. In embodiments, use of a compound according to Formula (I), or a pharmaceutically acceptable salt thereof, for decreasing activity of BCRP is provided which includes contacting cells expressing BCRP with the compound.

In embodiments, a compound according to Formula (I), or a pharmaceutically acceptable salt thereof, reduces multidrug resistance (MDR) in cancer chemotherapy utilized for treatment of the foregoing cancers. In embodiments, a compound according to Formula (I), or a pharmaceutically acceptable salt thereof, is administered in combination with a chemotherapeutic agent. In embodiments, the chemotherapeutic agent is a radiopharmaceutical agent. In embodiments, the chemotherapeutic agent is a photodynamic agent. In embodiments, administration of a compound according to Formula (I), or a pharmaceutically acceptable salt thereof, reduces metastasis of the foregoing cancers. In embodiments, administration of a compound according to Formula (I), or a pharmaceutically acceptable salt thereof, prevents metastasis of the foregoing cancers. In embodiments, administration of a compound according to Formula (I), or a pharmaceutically acceptable salt thereof, in combination with a chemotherapeutic agent reduces metastasis of the foregoing cancers. In embodiments, administration of a compound according to Formula (I), or a pharmaceutically acceptable salt thereof, in combination with a chemotherapeutic agent prevents metastasis of the foregoing cancers. In embodiments, a composition is provided which includes a compound according to Formula (I), or a pharmaceutically acceptable salt thereof, in combination with a chemotherapeutic agent.

In embodiments, an effective amount of a compound according to Formula (I), or a pharmaceutically acceptable salt thereof, is between about 0.001 mg/kg to about 500 mg/kg. In embodiments, about 0.5 mg/kg to about 250 mg/kg of a compound according to Formula (I), or a pharmaceutically acceptable salt thereof, is administered to a subject in need thereof. In embodiments, an effective amount of a compound according to Formula (I), or a pharmaceutically acceptable salt thereof, is administered to a subject once daily, twice daily, three times daily, four times daily, five times daily, six times daily, seven times daily or eight times daily. In embodiments, an effective amount of a compound according to Formula (I), or a pharmaceutically acceptable salt thereof, is administered every hour. In embodiments, an effective amount of a compound according to Formula (I), or a pharmaceutically acceptable salt thereof, is administered to a subject as a continuous infusion. In embodiments, an effective amount of a compound according to Formula (I), or a pharmaceutically acceptable salt thereof, is administered to a subject in combination with a chemotherapeutic agent. In embodiments, provided herein is a compound according to Formula (I), or a pharmaceutically acceptable salt thereof, for the manufacture of a medicament for treatment of cancer.

In embodiments, a compound according to Formula (I) is Compound A:

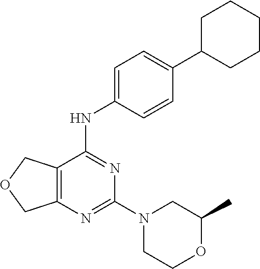 [SMILES: C[C@@H]1CN(c2nc3c(c(Nc4ccc(C5CCCCC5)cc4)n2)COC3)CCO1]

In embodiments, a compound according to Formula (I) is Compound B:

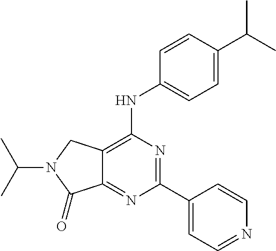 [SMILES: CC(C)c1ccc(Nc2nc(-c3ccncc3)nc3c2CN(C(C)C)C3=O)cc1]

In embodiments, a compound according to Formula (I) is Compound C:

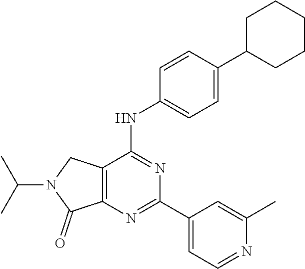 [SMILES: Cc1cc(-c2nc(Nc3ccc(C4CCCCC4)cc3)c3c(n2)C(=O)N(C(C)C)C3)ccn1]

In embodiments, a compound according to Formula (I) is Compound D:

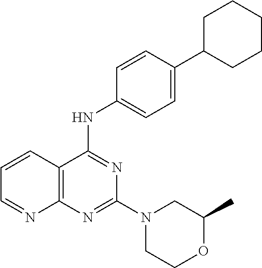 [SMILES: C[C@@H]1CN(c2nc(Nc3ccc(C4CCCCC4)cc3)c3cccnc3n2)CCO1]

In embodiments, a compound according to Formula (I) is Compound E:

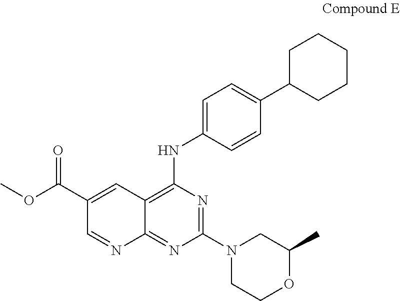 [SMILES: COC(=O)c1cnc2nc(N3CCO[C@H](C)C3)nc(Nc3ccc(C4CCCCC4)cc3)c2c1]

In embodiments, a compound according to Formula (I) is Compound F:

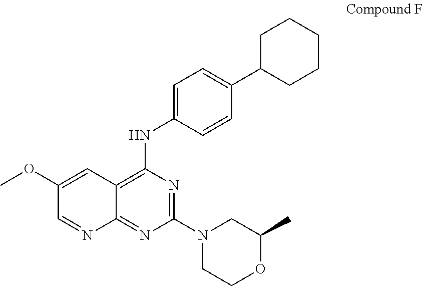 [SMILES: COc1cnc2nc(N3CCO[C@H](C)C3)nc(Nc3ccc(C4CCCCC4)cc3)c2c1]

In embodiments, a compound according to Formula (I) is Compound G:

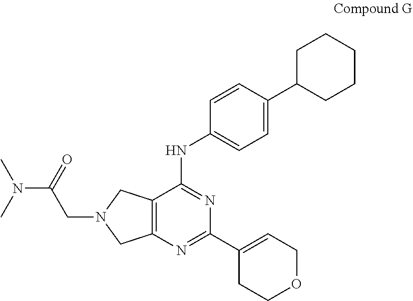 [SMILES: CN(C)C(=O)CN1Cc2nc(C3=CCOCC3)nc(Nc3ccc(C4CCCCC4)cc3)c2C1]

In embodiments, a compound according to Formula (I) is Compound H:

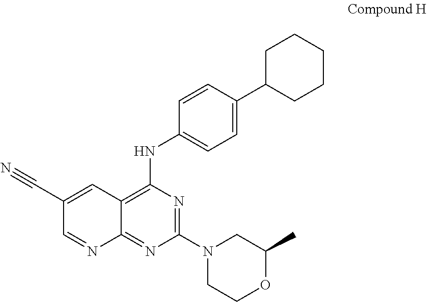 [SMILES: C[C@@H]1CN(c2nc(Nc3ccc(C4CCCCC4)cc3)c3cc(C#N)cnc3n2)CCO1]

### BRIEF DESCRIPTION OF THE DRAWINGS

FIG. 1 is a graph depicting Compound A inhibition of BCRP in a lipid vesicle assay (IC50=8 nM); ATP dependent accumulation percent of E3S versus concentration of Compound A.

FIG. 2 is a graph depicting Compound A inhibition of BCRP transporter efflux in cultured ASPC1 cancer cell lines.

FIG. 3 is a graph depicting Compound A inhibition of BCRP transporter efflux in cultured NCI-H2110-wt cancer cell lines.

FIG. 4 is a bar graph illustrating the effect of daily and weekly doses of Compound A on DS8201 treatment of CDx HT29 mice as measured by Tumor Volume Increase Over Baseline mm³.

FIG. 5 is a graph depicting the effect on tumor volume in HT-29 mice of vehicle alone, DS8201 3 mg/kg, and Compound A 5 mg/kg with DS8201 3 mg/kg over time.

FIG. 6 is a graph depicting the effect on tumor volume in HT-29 mice of vehicle alone, DS8201 10 mg/kg, and Compound A 5 mg/kg with DS8201 10 mg/kg over time.

### DETAILED DESCRIPTION

Methods and compositions for treating cancer are provided. In embodiments, methods and compositions for treating cancer are provided that reduce or prevent multidrug resistance (MDR) in cancer cells. In embodiments, methods for treating cancer include administering an effective amount of a compound according to Formula (I):

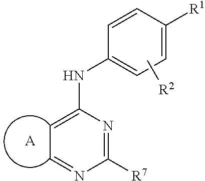

or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from

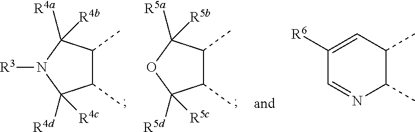

or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to a subject in need thereof.

In embodiments, compositions for treating cancer which include a compound according to Formula (I), or a pharmaceutically acceptable salt thereof, are administered to a subject in need thereof. A subject in need thereof may be a subject that has been diagnosed with cancer. In embodiments, the cancer is medulloblastoma, glioma, breast cancer, squamous cell cancer, melanoma, lung cancer, small cell lung cancer, cancer of the peritoneum, hepatocellular cancer, gastric cancer, colon cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia (CLL), acute lymphoblastic leukemia (ALL), hairy cell leukemia, leptomeningeal carcinomatosis, or chronic myeloblastic leukemia and subtypes thereof. In embodiments, an effective amount of a compound according to Formula (I), or a pharmaceutically acceptable salt thereof, is administered.

In embodiments, a compound according to Formula (I), or a pharmaceutically acceptable salt thereof, inhibits activity of breast cancer resistance protein (BCRP). In embodiments, a compound according to Formula (I), or a pharmaceutically acceptable salt thereof, reduces multidrug resistance (MDR) in cancer chemotherapy utilized for treatment of the foregoing cancers. In embodiments a compound according to Formula (I), or a pharmaceutically acceptable salt thereof, is administered in combination with a chemotherapeutic agent. In embodiments, the chemotherapeutic agent is a radiopharmaceutical agent. In embodiments, the chemotherapeutic agent is a photodynamic agent.

In embodiments, a method of decreasing activity of breast cancer resistance protein (BCRP) is provided which includes contacting cells expressing BCRP with a compound according to Formula (I), or a pharmaceutically acceptable salt thereof. In embodiments, use of a compound according to Formula (I), or a pharmaceutically acceptable salt thereof, for decreasing activity of BCRP is provided which includes contacting cells expressing BCRP with the compound.

In embodiments, a compound according to Formula (I), or a pharmaceutically acceptable salt thereof, reduces metastasis of the foregoing cancers. In embodiments a compound according to Formula (I), or a pharmaceutically acceptable salt thereof, prevents metastasis of the foregoing cancers. In embodiments, a compound according to Formula (I), or a pharmaceutically acceptable salt thereof, in combination with a chemotherapeutic agent reduces metastasis of the foregoing cancers. In embodiments, a compound according to Formula (I), or a pharmaceutically acceptable salt thereof, in combination with a chemotherapeutic agent prevents metastasis of the foregoing cancers. In embodiments, a composition is provided which includes a compound according to Formula (I), or a pharmaceutically acceptable salt thereof, in combination with a chemotherapeutic agent.

In embodiments, an effective amount of a compound according to Formula (I), or a pharmaceutically acceptable salt thereof, is between about 0.001 mg/kg to about 500 mg/kg. In embodiments, about 0.5 mg/kg to about 250 mg/kg of a compound according to Formula (I), or a pharmaceutically acceptable salt thereof, is administered to a subject in need thereof. A compound according to Formula (I), or a pharmaceutically acceptable salt thereof, will normally be administered to a subject, e.g., a warm-blooded animal at a unit dose within the range 2.5-5000 mg/m² body area of the animal, or approximately 0.05-500 mg/kg, and this normally provides a therapeutically-effective dose. Subjects” or “patients” include mammals, for example, humans. The terms “subjects” and “patients” may be used interchangeably herein. A unit dose form such as a tablet, capsule, film, patch, vial will can contain, for example 0.001-500 mg of a compound according to Formula (I), or a pharmaceutically acceptable salt thereof. In embodiments, an effective amount of a compound according to Formula (I), or a pharmaceutically acceptable salt thereof, is administered to a subject once daily, twice daily, three times daily, four times daily, five times daily, six times daily, seven times daily or eight or more times daily. In embodiments, an effective amount of a compound according to Formula (I), or a pharmaceutically acceptable salt thereof, is administered every hour. In embodiments, an effective amount of a compound according to Formula (I), or a pharmaceutically acceptable salt thereof, is administered to a subject as a continuous infusion. In embodiments, an effective amount of a compound according to Formula (I), or a pharmaceutically acceptable salt thereof, is administered to a subject in combination with a chemotherapeutic agent. In embodiments, provided herein is a compound according to Formula (I), or a pharmaceutically acceptable salt thereof, for the manufacture of a medicament for treatment of cancer.

The term “cancer”, as used herein refers to a proliferative disorder caused or characterized by a proliferation of cells which have lost susceptibility to normal growth control. Cancers of the same tissue type usually originate in the same tissue and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Non-limiting examples of cancers susceptible to treatment with a compound according to Formula (I), or a pharmaceutically acceptable salt thereof, include medulloblastoma, glioma, breast cancer, squamous cell cancer, melanoma, lung cancer, small cell lung cancer, cancer of the peritoneum, hepatocellular cancer, gastric cancer, colon cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, leptomeningeal carcinomatosis, or chronic myeloblastic leukemia and subtypes thereof, including refractory versions of any of the above cancers, or a combination of one or more of the above cancers.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. Leukemia is clinically detectable using one or more of complete blood counts, pallor, blood smears, and bone marrow smears. Advanced leukemias in certain subjects can manifest solid tumors.

Chemotherapy is well-known in the art and may be defined by the administration of numerous drugs and chemicals either alone or in combination to kill cancer cells. Chemotherapeutic agents kill cancer cells or reduce their progression.

As used herein, the term “BCRP substrate” refers to a drug or other compound whose efflux from a cell is mediated by BCRP. Methods of determining whether a given substance is a substrate for BCRP are known in the art. For example, efflux activity of BCRP may be evaluated by monitoring the basolateral-to-apical/apical-to-basolateral (B to A/A to B) efflux ratio of the compounds of interest in a cell or cell line expressing BCRP. See, e.g., Xia, C. Q., et al., Expression, localization and functional characteristics of breast cancer resistance protein in Caco-2 cells. Drug Met. Disp., 33 (5): 637-643 (2005).

BCRP substrates include chemotherapeutic agents. Examples of BCRP substrates may include, but are not limited to, methotrexate, mitoxantrone, imatinib, irinotecan, lapatinib, estrone-3-sulfate (E3 S), rosuvastatin, sulfasalazine, topotecan, daunorubicin, doxorubicin, epirubicin, bisatrene, flavpiridol, etoposide, teniposide 3, 9-aminocamptothecin, gefitinib, diflomotecan, zidovudine, rhodamine 123, SN-38. Chemotherapeutic agents include the foregoing, but are not limited to, abraxane, actinomycin, afatinib, alitretinoin, all-trans retinoic acid, altretamine, aminocamptothecin, amuvatinib, azacitidine, azathioprine, belotecan, bendamustine, bexarotene, bisatrene, bleomycin, bortezomib, busulfan, cabazitaxel, camptothecin, carboplatin, carboquone, carmustine, capecitabine, cisplatin, chlorambucil, chlormethine, chlorozotocin, cladribine, clofarabine, cyclophosphamide, cytarabine, dacarbazine, daunorubicin, decitabine, diflomotecan, docetaxel, doxifluridine, doxorubicin, epirubicin, epothilone, erlotinib, estrone-3-sulfate (E3 S), etoposide, exatecan, flavpiridol, fludarabine, fluorouracil, fotemustine, gefitinib, gemcitabine, gimatecan, hydroxyurea, idarubicin, ifosfamide, imatinib, irinotecan, ixabepilone, lapatinib, larotaxel, lomustine, melphalan, melphalan flufenamide, mercaptopurine, methotrexate, mitobronitol, mitomycin c, mitoxantrone, nelarabine, nimustine, nitrosoureas, oxaliplatin, paclitaxel, pemetrexed, pipobroman, ranimustine, rhodamine 123, romidepsin, rosuvastatin, semustine, SN-38, streptozotocin, sulfasalazine, tafluposide, taxotere, temozolomide, tesetaxel, teniposide, teniposide 3, 9-aminocamptothecin, thiotepa, tioguanine, topotecan, treosulfan, tretinoin, triaziquone, triethylenemelamine, valrubicin, vemurafenib, vinblastine, vincristine, vindesine, vinorelbine, vismodegib, vorinostat, and zidovudine. Chemotherapeutic agents include kinase inhibitors such as ABT-869, afatinib (BIBW-2992), AMG-706, AMN-107, amuvatinib, AST-487, axitinib (AG-013736), AZD-152HQPA, AZD-2171, BIBF-1120, BIRB-796, BMS-540215, bosutinib, cabozantinib, canertinib (Cl-1 033), CHIR-258/TKI-258, crizotinib, dasatinib, DMBI, dovitinib, erlotinib, everolimus, EXEL-2880/GSK-1363089, gefitinib, GW-786034, imatinib, JNJ-28312141, Ki-20227, Ki8751, lapatinib, masitinib (AB-1 01 0), midostaurin (PKC-412), motesanib, neratinib (HKI-272), nilotinib, OSI-930, pazopanib, PD-173955, PLX-4720, ponatinib, PTK-787, quizartinib (AC220), R406, regorafenib, SKI-606, sorafenib, staurosporine, SU-14813, sunitinib, tandutinib (MLN-518), telatinib, temsirolimus, tivozanib, vandetanib, vatalanib, and vemurafenib.

In embodiments, the term “effective amount” as applied to a compound according to Formula (I), or a pharmaceutically acceptable salt thereof, refers to an amount effective to prevent or treat cancer. In embodiments, an “effective amount” as applied to a compound according to Formula (I), or a pharmaceutically acceptable salt thereof, refers to an amount effective to inhibit effluxing activity of BCRP. In embodiments, an “effective amount” as applied to a compound according to Formula (I), or a pharmaceutically acceptable salt thereof, refers to an amount effective to overcome or reduce chemotherapeutic resistance in cancer cells. An “effective amount” of a compound according to Formula (I), or a pharmaceutically acceptable salt thereof, may be determined by various factors, for example, administration method, target site, the patient's condition, and the like. Therefore, the dosage when used in the human body should be determined in appropriate amounts in consideration of safety and efficacy. It is also possible to estimate the amount used in humans from the effective amount determined by animal experiments. See, e.g., Reagan-Shaw et al., FASEB J. 2008 March; 22(3):659-61 It should be understood that “inhibit” and “reduce” are used interchangeably herein.

The term “prevent”, including grammatical variants thereof such as “prevents”, “preventing” and “prevention” as used herein refers to actions that inhibit or delay the development, spread and recurrence of cancer by administration of a compound according to Formula (I), or a pharmaceutically acceptable salt thereof. The term “treat”, including grammatical variants thereof such as “treats”, “treating” and “treatment” as used herein refers to all types of actions that prevent or reduce the proliferation of cancer cells, facilitate the death of cancer cells and/or reduce symptoms of cancer by the administration a compound according to Formula (I), or a pharmaceutically acceptable salt thereof, according to the disclosure herein. “Treating cancer” herein includes inhibiting cancer proliferation and metastasis of cancer cells. For example, in embodiments, “treating” may include a reduction in seeding of metastatic cells or a reduction in seeding of tumor cells. As used herein, the terms “treat”, “treatment” or “treating” as applied to cancer encompass any manner in which the symptoms or pathology of a condition, disorder or disease associated with cancer are ameliorated or otherwise beneficially altered. In embodiments, “treat”, “treatment” or “treating” can refer to inhibiting a disease, disorder or condition, e.g., arresting or reducing its development or at least one clinical or subclinical symptom thereof. In embodiments, “treat”, “treatment” or “treating” can refer to relieving the disease, disorder or condition, e.g., causing regression of the disease, disorder or condition or at least one of its clinical or subclinical symptoms. The terms “treat”, “treatment” or “treating” may be used interchangeably herein. The benefit to a subject being treated may be statistically significant, mathematically significant, or at least perceptible to the subject and/or the physician.

The term “therapy” is intended to have its normal meaning of dealing with a disease in order to entirely or partially relieve one, some or all of its symptoms, or to correct or compensate for the underlying pathology. In embodiments, the term “therapy” may also include “prophylaxis” if “prophylaxis” is specifically referred to. The term “prophylaxis” is intended to have its normal meaning and includes primary prophylaxis to prevent the development of the disease and secondary prophylaxis whereby the disease or disorder has already developed and the patient is temporarily or permanently protected against exacerbation or worsening of the disease or the development of new symptoms associated with the disease. Nonetheless, prophylactic (preventive) and therapeutic treatment are two separate embodiments of the disclosure herein. In embodiments, the term “treatment” can be used synonymously with “therapy”. Similarly, the term “treat” can be regarded as “applying therapy” where “therapy” is as defined herein.

Many pharmaceutical products are administered as a fixed dose, at regular intervals, to achieve therapeutic efficacy. Duration of action is typically reflected by a drug's plasma half-life. Since efficacy is often dependent on sufficient exposure at the target site, e.g., tumor, bone marrow, etc., administration of a compound according to Formula (I), or a pharmaceutically acceptable salt thereof, having a short half-life may require frequent maintenance dosing. A compound according to Formula (I), or a pharmaceutically acceptable salt thereof, having a long half-life may require less frequent maintenance dosing.

It should be understood that in the context of Formula (I) and other Formulas disclosed herein that unless otherwise indicated, the term “alkyl” includes both linear and branched chain alkyl groups. The prefix C_{p-q} in C_{p-q} alkyl and other terms (where p and q are integers) indicates the range of carbon atoms that are present in the group, for example C₁₋₃alkyl includes C₁alkyl (methyl), C₂alkyl (ethyl) and C₃alkyl (propyl as n-propyl and isopropyl).

The term “C_{p-q} alkoxy” comprises —O—C_{p-q} alkyl groups and —C_{p-q} alkyl groups where the O atom is within the alkyl chain, for example, —CH₂—O—CH₃.

The term “C_{p-q} alkenyl” includes both linear and branched chain alkyl groups containing at least two carbon atoms and at least one double carbon-carbon bond.

The term “C_{p-q} alkenyloxy” comprises —O—C_{p-q} alkenyl groups and —C_{p-q} alkenyl groups where the O atom is within the alkenyl chain.

The term “C_{p-q} alkynyl” includes both linear and branched chain alkyl groups containing at least two carbon atoms and at least one triple carbon-carbon bond.

The term “C_{p-q} alkynyloxy” comprises —O—C_{p-q} alkynyl groups and —C_{p-q} alkynyl groups where the O atoms is within the alkynyl chain.

C_{p-q} cycloalkyl refers to a cyclic non-aromatic group of p-q carbon atoms and no heteroatoms. For example, a 3 to 7 membered cycloalkyl refers to a ring containing 3 to 7 carbon atoms. Suitable C₃₋₇cycloalkyl rings include cyclopropyl, cyclobutyl, cyclopentyl and cyclohexyl.

Aryl is a 6 to 10 membered monocyclic or bicyclic aromatic ring containing no heteroatoms. Aryl includes phenyl.

Heterocycloalkyl is a monocyclic saturated or partially unsaturated, non-aromatic ring having, for example, 3 to 7 members, such as 3 to 6 members, 5 to 7 members such as 5 or 6 members, where at least one member and up to 4 members, particularly 1, 2 or 3 members of the ring are heteroatoms selected from N, O and S, and the remaining ring atoms are carbon atoms, in stable combinations known to those of skill in the art. Heterocycloalkyl ring nitrogen and sulphur atoms are optionally oxidised. Suitable heterocycloalkyl rings include morpholinyl, thiazolidinyl, homomorpholine, tetrahydropyranyl, pyrrolyl, thiomorpholinyl and tetrahydrofuranyl. In embodiments, when R⁷ is heterocycloalkyl, optionally two substituents on the same ring carbon together with the carbon to which they are attached form a 5 to 7 membered heterocycloalkyl ring, thereby creating a spirocyclic ring system. For example, in embodiments, R⁷ is morpholinyl and two substituents on the same ring carbon together form a tetrahydropyran.

Heteroaryl is a polyunsaturated, monocyclic 5 or 6 membered aromatic ring containing at least one and up to 3 heteroatoms, particularly, 1 or 2 heteroatoms selected from N, O and S, and the remaining ring atoms are carbon atoms. Heteroaryl ring nitrogen and sulphur atoms are optionally oxidised. Suitable heteroaryl rings include pyridinyl, isoxazolyl, oxadiazolyl, imidazolyl, pyrazinyl, oxazolyl, thiophenyl and thiazolyl.

The term “halo” is fluorine, chlorine or bromine.

The use of the dashed bond “ [symbol] ” in rings A of Formula (I) represents the fusion of the pyrimidine ring.

Where the term “optionally” is used, it is intended that the subsequent feature may or may not occur. As such, use of the term “optionally” includes instances where the feature is present, and also instances where the feature is not present. For example, a group “optionally substituted with 1, 2 or 3 —F substituents” includes group with and without an —F substituent.

The term “substituted” means that one or more hydrogens (for example 1 or 2 hydrogens, or alternatively 1 hydrogen) on the designated group is replaced by the indicated substituent(s) (for example 1, 2 or 3 substituents, or alternatively 1 or 2 substituents, or alternatively 1 substituent), provided that any atom(s) bearing a substituent maintains a permitted valency. Substituent combinations encompass only stable compounds and stable synthetic intermediates. “Stable” means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e. that none of the hydrogens on the designated group have been replaced).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich:Wiley-VCH/VHCA, 2002. In embodiments, a suitable pharmaceutically acceptable salt of a compound according to the Formula (I) is, for example, a salt formed within the human or animal body after administration of a compound according to Formula (I), to said human or animal body.

In embodiments, a suitable pharmaceutically acceptable salt of a compound according to Formula (I) is, for example, an acid addition salt. An acid addition salt of a compound according to Formula (I) may be formed by bringing the compound into contact with a suitable inorganic or organic acid under conditions known to the skilled person. In embodiments, compounds described herein may form base addition salts. A base-addition salt of a compound according to Formula (I) may be formed by bringing the compound into contact with a suitable inorganic or organic base under conditions known to the skilled person.

In embodiments, a compound according to Formula (I) may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

In embodiments, there is provided a compound according to Formula (I), or a pharmaceutically acceptable salt thereof. In embodiments, there is provided a compound according to Formula (I). In embodiments there is provided a pharmaceutically acceptable salt of a compound according to Formula (I).

Compounds and salts described herein may exist in solvated forms and unsolvated forms. For example, a solvated form may be a hydrated form, such as a hemi-hydrate, a monohydrate, a dihydrate, a trihydrate or an alternative quantity thereof. The description herein encompasses all such solvated and unsolvated forms of compounds of Formula (I), particularly to the extent that such forms possess KCC2 modulating activity, as for example measured using the tests described herein.

The following embodiments of moiety A may be applied to the description of the compounds of Formula (I), provided herein:
A is selected from:

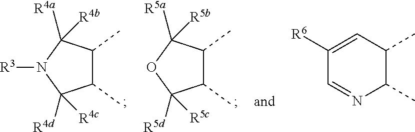

or a N-oxide thereof.

In embodiments, A is

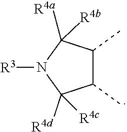

In embodiments, A is

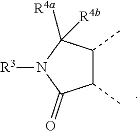

In embodiments, A is

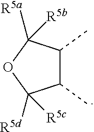

In embodiments, A is

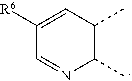

In embodiments, A is

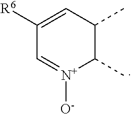

In embodiments, there is provided a compound according to Formula (II):

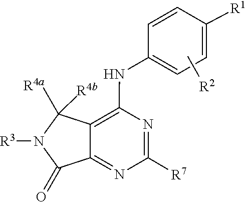

or a pharmaceutically acceptable salt thereof, wherein R¹, R², R³, R^{4a}, R^{4b} and R⁷ are as defined for Formula (I).

In embodiments, there is provided a compound according to Formula (II) or a pharmaceutically acceptable salt thereof, wherein R¹, R², R³, R^{4a}, R^{4b} and R⁷ are as defined for Formula (I) and when R⁷ is morpholinyl, either:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃; heteroaryl is optionally substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and aryl is substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and R² is selected from —H; -halo; and —C₁₋₃ alkyl optionally substituted with 1, 2 or 3 substituents selected from —F and —CF₃; or
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃; and heteroaryl is optionally substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and R² is selected from -halo and —C₁₋₃alkyl optionally substituted with 1, 2 or 3 substituents selected from —F and —CF₃.

In embodiments, there is provided a compound according to Formula (II) or a pharmaceutically acceptable salt thereof, wherein:
R¹ is selected from —C₂₋₆alkyl; —C₂₋₆alkoxy; C₃₋₇cycloalkyl; —O—C₃₋₇cycloalkyl; phenyl optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; —O-phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃;
R² is selected from —H, —F and —CH₃;
R³ is selected from —C₂₋₄alkynyl and —C₁₋₃alkyl optionally substituted with —NR⁸R⁹;
R^{4a} and R^{4b} are both —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1 or 2 substituents selected from —CN; —C₁₋₃alkyl optionally substituted with 1, 2 or 3 substituents selected from —F, —CF₃ and —OH; —C₁₋₃alkoxy; cyclopropyl; —C(O)OH; —C₁₋₃alkylene-NHC(O)C₁₋₆alkyl; —C₁₋₃alkylene-NHC(O)OC₁₋₆alkyl; or the heterocycloalkyl is optionally substituted with two substituents on the same ring carbon which together with the carbon atom to which they are attached form a 6 membered monocyclic heterocycloalkyl;
R⁸ and R⁹ are each independently selected from —C₁₋₆alkyl;
R¹⁰ is selected from —C₁₋₃alkyl;
R¹¹ is selected from —C₁₋₃alkyl optionally substituted with 1 or 2 substituents selected from —F and —C₁₋₃alkoxy; and —(CH₂)ₙR¹²;
R¹² is selected from a 5 or 6 membered heteroaryl, a 3 to 5 membered cycloalkyl or a 3 to 6 membered heterocycloalkyl;
n is 1 or 2.

In embodiments, there is provided a compound according to Formula (II) or a pharmaceutically acceptable salt thereof, wherein:
R¹ is selected from —C₂₋₆alkyl; —C₂₋₆alkoxy; C₃₋₇cycloalkyl; —O—C₃₋₇cycloalkyl; phenyl substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; —O-phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃; and R² is selected from —H, —F and —CH₃; or
R¹ is selected from —C₂₋₆alkyl; —C₂₋₆alkoxy; C₃₋₇cycloalkyl; —O—C₃₋₇cycloalkyl; unsubstituted phenyl; —O-phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃; and R² is selected from —F and —CH₃;
R³ is selected from —C₂₋₄alkynyl and —C₁₋₃alkyl optionally substituted with —NR⁸R⁹;
R^{4a} and R^{4b} are both —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1 or 2 substituents selected from —CN; —C₁₋₃alkyl optionally substituted with 1, 2 or 3 substituents selected from —F, —CF₃ and —OH; —C₁₋₃alkoxy; cyclopropyl; —C(O)OH; —C₁₋₃alkylene-NHC(O)C₁₋₆alkyl; —C₁₋₃alkylene-NHC(O)OC₁₋₆alkyl; or the heterocycloalkyl is optionally substituted with two substituents on the same ring carbon which together with the carbon atom to which they are attached form a 6 membered monocyclic heterocycloalkyl;
R⁸ and R⁹ are each independently selected from —C₁₋₆alkyl;
R¹⁰ is selected from —C₁₋₃alkyl;
R¹¹ is selected from —C₁₋₃alkyl optionally substituted with 1 or 2 substituents selected from —F and —C₁₋₃alkoxy; and —(CH₂)ₙR¹²;
R¹² is selected from a 5 or 6 membered heteroaryl, a 3 to 5 membered cycloalkyl or a 3 to 6 membered heterocycloalkyl; and
n is 1 or 2.

In embodiments, there is provided a compound according to Formula (III):

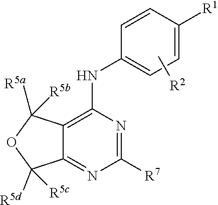

or a pharmaceutically acceptable salt thereof, wherein R¹, R², R^{5a}, R^{5b}, R^{5c}, R^{5d} and R⁷ are as defined for Formula (I).

In embodiments, there is provided a compound according to Formula (III) or a pharmaceutically acceptable salt thereof, wherein:
R¹ is selected from C₃₋₇cycloalkyl and C₆₋₁₀aryl, wherein the aryl is optionally substituted with a —C₂₋₈alkoxy substituent wherein the alkoxy is optionally substituted with 1 or 2 —CF₃ substituents;
R² is —H;
R^{5a}, R^{5b}, R^{5c} and R^{5d} are each —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H.

In embodiments, there is provided a compound according to Formula (III) or a pharmaceutically acceptable salt thereof, wherein:
R¹ is selected from C₃₋₇cycloalkyl and C₆₋₁₀aryl, wherein the aryl is optionally substituted with a —C₂₋₈alkoxy substituent wherein the alkoxy is optionally substituted with 1 or 2 —CF₃ substituents;
R² is —H;
R^{5a}, R^{5b}, R^{5c} and R^{5d} are each —H;
R⁷ is selected from a 5 to 7 membered monocyclic heterocycloalkyl and a 5 or 6 membered monocyclic heteroaryl, wherein the heterocycloalkyl and heteroaryl are optionally substituted with a substituent selected from —C₁₋₃alkyl optionally substituted with 1, 2 or 3 substituents selected from —F and —OH; and cyclopropyl.

In embodiments, there is provided a compound according to Formula (IV):

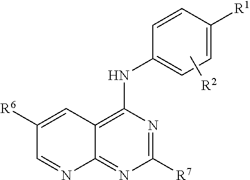

or a N-oxide or pharmaceutically acceptable salt thereof, wherein R¹, R², R⁶ and R⁷ are as defined for Formula (I).

In embodiments, there is provided a compound according to Formula (IV), or a N-oxide or pharmaceutically acceptable salt thereof, wherein:
R¹ selected from C₃₋₇cycloalkyl optionally substituted with 1, 2 or 3 substituents selected from —F and —CF₃;
R² is selected from —H; -halo; and —C₁₋₃alkyl optionally substituted with 1, 2 or 3 substituents selected from —F and —CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
n is 1, 2 or 3.

In embodiments, there is provided a compound according to Formula (IV), or a N-oxide or pharmaceutically acceptable salt thereof, wherein:
R¹ selected from C₃₋₇cycloalkyl optionally substituted with 1, 2 or 3 substituents selected from —F and —CF₃;
R² is —H;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from a 5 to 7 membered monocyclic heterocycloalkyl optionally substituted with 1, 2 or 3 groups selected from —C₁₋₆alkyl optionally substituted with 1, 2 or 3 substituents selected from —F, —CF₃ and —OH; and C₃₋₅cycloalkyl;
R⁸ and R⁹ are each independently selected from —H and —C₁₋₆alkyl.

The following embodiments of moieties R¹, R², R³, R^{4a}, R^{4b}, R^{4c}, R^{4d}, R⁵, R^{6a}, R^{6b}, R⁷, R⁸, R⁹, R¹⁰, R¹¹, R¹², m and n may be applied, alone or in combination, to the description of the compounds of Formula (I) provided herein. The following embodiments of moieties R¹, R², R³, R^{4a}, R^{4b}, R⁷, R⁸, R⁹, R¹⁰, R¹¹, R¹², m and n may be applied, alone or in combination, to the description of the compounds of Formula (II) provided herein. The following embodiments of moieties R¹, R², R^{5a}, R^{5b}, R^{5c}, R^{5d}, R⁷, R⁸, R⁹, R¹⁰, R¹¹, R¹², m and n may be applied, alone or in combination, to the description of the compounds of Formula (III) provided herein. The following embodiments of moieties R¹, R², R⁶, R⁷, R⁸, R⁹, R¹⁰, R¹¹, R¹², m and n may be applied, alone or in combination, to the descriptions of the compounds of Formula (IV) provided herein.

R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein —O—(CH₂)ₘ—C₆₋₁₀aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl; and C₆₋₁₀aryl is substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl

In embodiments, R¹ is selected from C₂₋₆alkyl; C₂₋₆alkoxy; C₃₋₇cycloalkyl; —O—C₃-7cycloalkyl; C₆₋₁₀aryl; —O—(CH₂)ₘ—C₆₋₁₀aryl and thiophenyl; wherein alkyl, alkoxy and cycloalkyl are optionally substituted with 1, 2 or 3 substituents selected from —F and —CF₃ and wherein aryl is optionally substituted with 1 or 2 substituents selected from -halo, —C₁₋₃alkyl, —C₁₋₈alkoxy and —C₂-8alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl optionally substituted with 1, 2 or 3 substituents selected from —F and —CF₃; C₂₋₄alkoxy; C₄₋₆cycloalkyl; —O—C₄₋₆cycloalkyl; phenyl; —O—(CH₂)ₘ-phenyl; and thiophenyl; wherein phenyl is optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl optionally substituted with 1, 2 or 3 substituents selected from —F and —CF₃; C₂₋₄alkoxy; C₄₋₆cycloalkyl; —O—C₄₋₆cycloalkyl; phenyl; —O—(CH₂)ₘ-phenyl; and thiophenyl; wherein —O—(CH₂)ₘ-phenyl is optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl; and phenyl is substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from —CF₂CF₃; propyl; butyl; pentyl; propoxy; cyclobutyl; cyclohexyl; —O-cyclopentyl; thiophenyl; phenyl; —O-phenyl; —O—CH₂-phenyl; wherein phenyl is optionally substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇.

In embodiments, R¹ is selected from —CF₂CF₃; propyl; butyl; pentyl; propoxy; cyclobutyl; cyclohexyl; —O-cyclopentyl; thiophenyl; phenyl substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇; —O-phenyl; —O—CH₂-phenyl; wherein —O-phenyl and —O—CH₂-phenyl is optionally substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇.

In embodiments, R¹ is cyclohexyl. In another embodiment, R¹ is phenyl substituted with —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇. In embodiments, R¹ is phenyl.

R² is selected from —H, -halo and —C₁₋₃alkyl optionally substituted with 1, 2 or 3 substituents selected from —F and —CF₃. In embodiments, R² is —H. In embodiments, R² is -halo. In embodiments, R² is —F. In embodiments, R² is —C₁₋₃alkyl. In embodiments, R² is methyl.

R³ is selected from —H; —C₁₋₆alkyl; —C₂₋₆alkenyl; —C₂₋₆alkynyl; —C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups, for example 1 or 2 groups, selected from —C₁₋₃alkyl optionally substituted with 1, 2 or 3 substituents selected from —F, —CF₃, —C(O)NR⁸R⁹ and —NR⁸R⁹.

In embodiments, R³ is selected from —H; —C₂₋₄alkynyl; —C₁₋₃alkyl optionally substituted with —C(O)NR⁸R⁹ or —NR⁸R⁹; and a 5 or 6 membered heterocycloalkyl optionally substituted with C₁₋₃alkyl.

In embodiments, R³ is selected from —H; —C₂₋₄alkynyl; —C₁₋₃alkyl optionally substituted with —C(O)NR⁸R⁹ or —NR⁸R⁹; and a 5 or 6 membered nitrogen containing heterocycloalkyl optionally substituted with C₁₋₃alkyl.

In embodiments, R³ is selected from —H; —C₂₋₄alkynyl; —C₁₋₃alkyl optionally substituted with —C(O)NR⁸R⁹ or —NR⁸R⁹; and piperidinyl optionally substituted with C₁₋₃alkyl.

In embodiments, R³ is selected from methyl, ethyl, i-propyl, —(CH₂)₂N(CH₃)₂, —(CH₂)₃N(CH₃)₂, —CH₂C≡CH, —CH₂C(O)N(CH₃)₂ and N-methylpiperidine. In embodiments, R³ is selected from ethyl, i-propyl, —(CH₂)₂N(CH₃)₂, —(CH₂)₃N(CH₃)₂, —CH₂C≡CH, —CH₂C(O)N(CH₃)₂ and N-methylpiperidine.

In embodiments, R³ is selected from —C₂₋₄alkynyl and —C₁₋₃alkyl optionally substituted with —NR⁸R⁹.

In embodiments, R³ is selected from ethyl, i-propyl, —(CH₂)₂N(CH₃)₂, —(CH₂)₃N(CH₃)₂ and —CH₂C≡CH.

In embodiments, R³ is i-propyl.

R^{4a} and R^{4b} are each independently selected from —H and —C₁₋₃alkyl optionally substituted with 1, 2 or 3 substituents selected from —F and —CF₃. In embodiments, R^{4a} is methyl and R^{4b} is —H. In embodiments, R^{4a} and R^{4b} are both —H.

R^{4c} and R^{4d} are each independently selected from —H and C₁₋₃ alkyl optionally substituted with 1, 2 or 3 substituents selected from —F and —CF₃; or R^{4c} and R^{4d} together with the carbon to which they are attached represent carbonyl. In embodiments, R^{4c} and R^{4d} together with the carbon to which they are attached represent carbonyl. In embodiments, R^{4c} and R^{4d} are each independently selected from —H and C₁₋₃ alkyl optionally substituted with 1, 2 or 3 substituents selected from —F and —CF₃. In embodiments, R^{4c} and R^{4d} are both —H or together with the carbon to which they are attached represent carbonyl. In embodiments, R^{4c} and R^{4d} are both —H.

R^{5a}, R^{5b}, R^{5c} and R^{5d} are each independently selected from —H and —C₁₋₃ alkyl optionally substituted with 1, 2 or 3 substituents selected from —F and —CF₃. In embodiments, R^{5a}, R^{5b}, R^{5c} and R^{5d} are each independently selected from —H and —C₁₋₃ alkyl optionally substituted with 1, 2 or 3 substituents selected from —F and —CF₃. In embodiments, R^{5a}, R^{5b}, R^{5c} and R^{5d} are each independently selected from —H and —C₁₋₃ alkyl. In embodiments, R^{5a} is methyl and R^{5b}, R^{5c} and R^{5d} are each —H. In embodiments, R^{5a}, R^{5b} and R^{5c} are each —H and R^{5d} is methyl. In embodiments, R^{5a}, R^{5b}, R^{5c} and R^{5d} each represent —H.

R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl. In embodiments, R⁶ is selected from —H; —Br; —NH₂; —CN; methoxy; ethyl; —C(O)OCH₃; —C(O)NH₂; —C(O)OH; and —NHC(O)CH₃.

R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl.

In embodiments, R⁷ is selected from NR¹⁰R¹¹; a 5 to 7 membered monocylic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl.

In embodiments, R⁷ is selected from NR¹⁰R¹¹; a 5 to 7 membered monocylic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1, 2 or 3 (for example, 1 or 2) groups selected from —CN, methyl, ethyl, propyl, cyclopropyl, methoxy, —CH₂CF₃, —CH₂OH, —CH₂CH₂OH, —C(O)OH, —(CH₂)₂NHC(O)CH₃ and —CH₂NHC(O)OC(CH₃)₃; or the heterocycloalkyl is optionally substituted with two substituents on the same ring carbon which together with the carbon atom to which they are attached form a 6 membered monocyclic heterocycloalkyl.

In embodiments, R⁷ is selected from NR¹⁰R¹¹ wherein R¹⁰ is selected from methyl, ethyl or propyl and R¹¹ is selected from ethyl, propyl, CH₂CHF₂, CH₂CH₂OCH₂CH₃ and —(CH₂)ₚR¹²; a 5 to 7 membered monocyclic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1 or 2 groups selected from —CN, methyl, ethyl, propyl, cyclopropyl, methoxy, —CH₂CF₃, —CH₂OH, —CH₂CH₂OH, —C(O)OH, —(CH₂)₂NHC(O)CH₃ and —CH₂NHC(O)OC(CH₃)₃; or the heterocycloalkyl is optionally substituted with two substituents on the same ring carbon which together with the carbon atom to which they are attached form tetrahydropyranyl.

In embodiments, R⁷ is selected from NR¹⁰R¹¹ wherein R¹⁰ is selected from methyl, ethyl or propyl and R¹¹ is selected from ethyl, propyl, CH₂CHF₂, CH₂CH₂OCH₂CH₃ and —(CH₂)ₙR¹².

In embodiments, R⁷ is selected from NR¹⁰R¹¹ wherein R¹⁰ is selected from methyl, ethyl or propyl; R¹¹ is selected from ethyl, propyl, CH₂CHF₂, CH₂CH₂OCH₂CH₃ and —(CH₂)ₙR¹²; n is 1 or 2; and R¹² is selected from isoxazolyl, oxadiazolyl, cyclopropyl, pyrazinyl, tetrahydrofuranyl and pyridinyl.

In embodiments, R⁷ is selected from a 5 to 7 membered monocyclic heterocycloalkyl optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃ alkylene-NHC(O)C₁₋₆alkyl; —C₁₋₃alkylene-NHC(O)OC₁₋₆alkyl and C₃₋₅cycloalkyl; or the heterocycloalkyl is optionally substituted with two substituents on the same ring carbon which together with the carbon atom to which they are attached form a 5 to 7 membered monocyclic heterocycloalkyl.

In embodiments, R⁷ is a 5 to 7 membered monocyclic heterocycloalkyl selected from morpholinyl, thiazolidinyl, tetrahydropyranyl, pyrrolyl, thiomorpholinyl and 3,4-dihydro-2H-pyranyl wherein the heterocycloalkyl is optionally substituted with 1 or 2 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —CH₂NHC(O)CH₃; —CH₂NHC(O)OC(CH₃)₃; and C₃₋₅cycloalkyl; or the heterocycloalkyl is optionally substituted with two substituents on the same ring carbon which together with the carbon atom to which they are attached form a 6 membered monocyclic heterocycloalkyl.

In embodiments, R⁷ is a 5 to 7 membered monocyclic heterocyclalkyl optionally substituted with 1 or 2 substituents selected from methyl, ethyl, propyl, cyclopropyl, —CH₂CH₂OH, —CH₂OH, —C(O)OH, —CH₂CF₃, and —CH₂NHC(O)OC(CH₃)₃; or the heterocycloalkyl is optionally substituted with two substituents on the same ring carbon which together with the carbon atom to which they are attached form tetrahydropyran.

In embodiments, R⁷ is morpholinyl optionally substituted with 1 or 2 substituents selected from methyl, ethyl, propyl, cyclopropyl, —CH₂CH₂OH, —CH₂OH, —C(O)OH, —CH₂CF₃, and —CH₂NHC(O)OC(CH₃)₃; or optionally substituted with two substituents on the same ring carbon which together with the carbon atom to which they are attached form tetrahydropyran (i.e. R⁷ becomes a spirocyclic group).

In embodiments, R⁷ is 2-methylmorpholin-4-yl.

R⁸ is selected from —H and —C₁₋₆alkyl. In embodiments, R⁸ is selected from —H and —C₁₋₃ alkyl. In embodiments, R⁸ is —H. In embodiments, R⁸ is —C₁₋₃alkyl. In embodiments, R⁸ is methyl.

R⁹ is selected from —H and —C₁₋₆alkyl. In embodiments, R⁹ is selected from —H and —C₁₋₃ alkyl. In embodiments, R⁹ is —H. In embodiments, R⁹ is —C₁₋₃alkyl. In embodiments, R⁹ is methyl.

R¹⁰ is —C₁₋₆alkyl. In embodiments, R¹⁰ is —C₁₋₃alkyl. In embodiments, R¹⁰ is methyl. In another embodiment, R¹⁰ is ethyl. In embodiments, R¹⁰ is propyl.

R¹¹ is selected from —C₁₋₆alkyl optionally substituted with 1 or 2 substituents selected from —F and —C₁₋₃alkoxy; or —(CH₂)ₙR¹². In embodiments, R¹¹ is selected from —C₁₋₆alkyl optionally substituted with 1 or 2 substituents selected from —F and ethoxy. In embodiments, R¹¹ is selected from ethyl, propyl, CH₂CHF₂, CH₂CH₂OCH₂CH₃ and —(CH₂)ₙR¹². In embodiments, R¹¹ is selected from —(CH₂)ₙR¹².

R¹² is selected from a 5 or 6 membered heteroaryl, a 3 to 5 membered cycloalkyl or a 3 to 6 membered heterocycloalkyl. In embodiments, R¹² is selected from isoxazolyl, oxadiazolyl, cyclopropyl, pyrazinyl, tetrahydrofuranyl and pyridinyl.

m is 0 or 1. In embodiments, m is 0. In embodiments, m is 1.

n is 1, 2 or 3. In embodiments, n is 1 or 2. In embodiments, n is 1. In embodiments, n is 2. In embodiments, n is 3.

In embodiments, a compound according to Formula (I) is selected from:
2-(diethylamino)-6-(propan-2-yl)-4-{[4-(propan-2-yl)phenyl]amino}-5,6-dihydro-7H-pyrrolo[3,4-d]pyrimidin-7-one;
4-[(4-cyclohexylphenyl)amino]-2-(2-cyclopropylmorpholin-4-yl)-6-(propan-2-yl)-5,6-dihydro-7H-pyrrolo[3,4-d]pyrimidin-7-one;
6-(propan-2-yl)-4-{[4-(propan-2-yl)phenyl]amino}-2-(1,3-thiazolidin-3-yl)-5,6-dihydro-7H-pyrrolo[3,4-d]pyrimidin-7-one;
2-[(2R,6S)-2,6-dimethylmorpholin-4-yl]-6-(propan-2-yl)-4-{[4-(propan-2-yl)phenyl]amino}-5,6-dihydro-7H-pyrrolo[3,4-d]pyrimidin-7-one;
6-(propan-2-yl)-4-{[4-(propan-2-yl)phenyl]amino}-2-(thiomorpholin-4-yl)-5,6-dihydro-7H-pyrrolo[3,4-d]pyrimidin-7-one;
2-[(2S)-2-methylmorpholin-4-yl]-6-(propan-2-yl)-4-{[4-(propan-2-yl)phenyl]amino}-5,6-dihydro-7H-pyrrolo[3,4-d]pyrimidin-7-one;
2-[(2R)-2-methylmorpholin-4-yl]-6-(propan-2-yl)-4-{[4-(propan-2-yl)phenyl]amino}-5,6-dihydro-7H-pyrrolo[3,4-d]pyrimidin-7-one;
2-[(2S,6S)-2,6-dimethylmorpholin-4-yl]-6-(propan-2-yl)-4-{[4-(propan-2-yl)phenyl]amino}-5,6-dihydro-7H-pyrrolo[3,4-d]pyrimidin-7-one;
2-(3-methylmorpholin-4-yl)-6-(propan-2-yl)-4-{[4-(propan-2-yl)phenyl]amino}-5,6-dihydro-7H-pyrrolo[3,4-d]pyrimidin-7-one;
2-(2-cyclopropylmorpholin-4-yl)-6-(propan-2-yl)-4-{[4-(propan-2-yl)phenyl]amino}-5,6-dihydro-7H-pyrrolo[3,4-d]pyrimidin-7-one;
4-[(4-cyclohexylphenyl)amino]-2-(morpholin-4-yl)-6-(propan-2-yl)-5,6-dihydro-7H-pyrrolo[3,4-d]pyrimidin-7-one;
4-[(4-cyclohexylphenyl)amino]-2-(2-methylmorpholin-4-yl)-6-(propan-2-yl)-5,6-dihydro-7H-pyrrolo[3,4-d]pyrimidin-7-one;
4-[(4-cyclohexylphenyl)amino]-2-[(2R)-2-methylmorpholin-4-yl]-6-(propan-2-yl)-5,6-dihydro-7H-pyrrolo[3,4-d]pyrimidin-7-one;
4-[(4-cyclohexylphenyl)amino]-2-((2R)-cyclopropylmorpholin-4-yl)-6-(propan-2-yl)-5,6-dihydro-7H-pyrrolo[3,4-d]pyrimidin-7-one;
4-[(4-cyclohexylphenyl)amino]-2-((2S)-cyclopropylmorpholin-4-yl)-6-(propan-2-yl)-5,6-dihydro-7H-pyrrolo[3,4-d]pyrimidin-7-one;
4-[(4-cyclohexylphenyl)amino]-6-(propan-2-yl)-2-[2-(2,2,2-trifluoroethyl)morpholin-4-yl]-5,6-dihydro-7H-pyrrolo[3,4-d]pyrimidin-7-one;
tert-butyl {[(2R)-4-{4-[(4-cyclohexylphenyl)amino]-7-oxo-6-(propan-2-yl)-6,7-dihydro-5H-pyrrolo[3,4-d]pyrimidin-2-yl}morpholin-2-yl]methyl}carbamate;
4-[(4-cyclohexylphenyl)amino]-6-(propan-2-yl)-2-[2-(propan-2-yl)morpholin-4-yl]-5,6-dihydro-7H-pyrrolo[3,4-d]pyrimidin-7-one;
4-[(4-cyclohexylphenyl)amino]-6-(propan-2-yl)-2-(1,3-thiazolidin-3-yl)-5,6-dihydro-7H-pyrrolo[3,4-d]pyrimidin-7-one;
4-[(4-cyclohexylphenyl)amino]-2-[(2-ethoxyethyl)(methyl)amino]-6-(propan-2-yl)-5,6-dihydro-7H-pyrrolo[3,4-d]pyrimidin-7-one;
4-[(4-cyclohexylphenyl)amino]-2-(2-ethylmorpholin-4-yl)-6-(propan-2-yl)-5,6-dihydro-7H-pyrrolo[3,4-d]pyrimidin-7-one;
4-[(4-cyclohexylphenyl)amino]-2-{methyl[(1,2-oxazol-3-yl)methyl]amino}-6-(propan-2-yl)-5,6-dihydro-7H-pyrrolo[3,4-d]pyrimidin-7-one;
4-[(4-cyclohexylphenyl)amino]-2-{methyl[2-(1,2,4-oxadiazol-3-yl)ethyl]amino}-6-(propan-2-yl)-5,6-dihydro-7H-pyrrolo[3,4-d]pyrimidin-7-one;
4-[(4-cyclohexylphenyl)amino]-2-(1,4-oxazepan-4-yl)-6-(propan-2-yl)-5,6-dihydro-7H-pyrrolo[3,4-d]pyrimidin-7-one;
4-[(4-cyclohexylphenyl)amino]-2-(1,9-dioxa-4-azaspiro[5.5]undecan-4-yl)-6-(propan-2-yl)-5,6-dihydro-7H-pyrrolo[3,4-d]pyrimidin-7-one;
4-[(4-cyclohexylphenyl)amino]-2-(3-methoxypyrrolidin-1-yl)-6-(propan-2-yl)-5,6-dihydro-7H-pyrrolo[3,4-d]pyrimidin-7-one;
4-[(4-cyclohexylphenyl)amino]-2-[2-(2-hydroxyethyl)morpholin-4-yl]-6-(propan-2-yl)-5,6-dihydro-7H-pyrrolo[3,4-d]pyrimidin-7-one;
4-[(4-cyclohexylphenyl)amino]-2-(dipropylamino)-6-(propan-2-yl)-5,6-dihydro-7H-pyrrolo[3,4-d]pyrimidin-7-one;
4-[(4-cyclohexylphenyl)amino]-2-[(cyclopropylmethyl)(methyl)amino]-6-(propan-2-yl)-5,6-dihydro-7H-pyrrolo[3,4-d]pyrimidin-7-one;
4-[(4-cyclohexylphenyl)amino]-2-[2-(hydroxymethyl)morpholin-4-yl]-6-(propan-2-yl)-5,6-dihydro-7H-pyrrolo[3,4-d]pyrimidin-7-one;
4-[(4-cyclohexylphenyl)amino]-2-[3-(hydroxymethyl)morpholin-4-yl]-6-(propan-2-yl)-5,6-dihydro-7H-pyrrolo[3,4-d]pyrimidin-7-one;
4-[(4-cyclohexylphenyl)amino]-2-{methyl[(pyrazin-2-yl)methyl]amino}-6-(propan-2-yl)-5,6-dihydro-7H-pyrrolo[3,4-d]pyrimidin-7-one;
4-[(4-cyclohexylphenyl)amino]-2-(diethylamino)-6-(propan-2-yl)-5,6-dihydro-7H-pyrrolo[3,4-d]pyrimidin-7-one;
4-[(4-cyclohexylphenyl)amino]-2-{methyl[(oxolan-2-yl)methyl]amino}-6-(propan-2-yl)-5,6-dihydro-7H-pyrrolo[3,4-d]pyrimidin-7-one;
4-[(4-cyclohexylphenyl)amino]-2-[(2,2-difluoroethyl)(methyl)amino]-6-(propan-2-yl)-5,6-dihydro-7H-pyrrolo[3,4-d]pyrimidin-7-one;
4-[(4-cyclohexylphenyl)amino]-2-{methyl[2-(pyridin-2-yl)ethyl]amino}-6-(propan-2-yl)-5,6-dihydro-7H-pyrrolo[3,4-d]pyrimidin-7-one;
(3S)-4-{4-[(4-cyclohexylphenyl)amino]-7-oxo-6-(propan-2-yl)-6,7-dihydro-5H-pyrrolo[3,4-d]pyrimidin-2-yl}morpholine-3-carboxylic acid;
N-[2-(4-{4-[(4-cyclohexylphenyl)amino]-7-oxo-6-(propan-2-yl)-6,7-dihydro-5H-pyrrolo[3,4-d]pyrimidin-2-yl}morpholin-2-yl)ethyl]acetamide;
6-(propan-2-yl)-4-{[4-(propan-2-yl)phenyl]amino}-2-(pyridin-4-yl)-5,6-dihydro-7H-pyrrolo[3,4-d]pyrimidin-7-one;
6-isopropyl-4-((4-isopropylphenyl)amino)-2-(pyridin-4-yl)-5,6-dihydro-7H-pyrrolo[3,4-d]pyrimidin-7-one (Compound B);
4-{4-[(4-cyclohexylphenyl)amino]-7-oxo-6-(propan-2-yl)-6,7-dihydro-5H-pyrrolo[3,4-d]pyrimidin-2-yl}pyridine-2-carbonitrile;
4-[(4-cyclohexylphenyl)amino]-2-(2-cyclopropylpyridin-4-yl)-6-(propan-2-yl)-5,6-dihydro-7H-pyrrolo[3,4-d]pyrimidin-7-one;
4-[(4-cyclohexylphenyl)amino]-2-(2-methoxypyridin-4-yl)-6-(propan-2-yl)-5,6-dihydro-7H-pyrrolo[3,4-d]pyrimidin-7-one;
4-[(4-cyclohexylphenyl)amino]-2-(2-methylpyridin-4-yl)-6-(propan-2-yl)-5,6-dihydro-7H-pyrrolo[3,4-d]pyrimidin-7-one (Compound C);
4-[(4-cyclohexylphenyl)amino]-2-(3,6-dihydro-2H-pyran-4-yl)-6-(propan-2-yl)-5,6-dihydro-7H-pyrrolo[3,4-d]pyrimidin-7-one;
4-[(4-cyclohexylphenyl)amino]-6-(propan-2-yl)-2-(pyridin-4-yl)-5,6-dihydro-7H-pyrrolo[3,4-d]pyrimidin-7-one;
4-[(4-cyclohexylphenyl)amino]-2-(1-methyl-1H-pyrazol-4-yl)-6-(propan-2-yl)-5,6-dihydro-7H-pyrrolo[3,4-d]pyrimidin-7-one;
4-[(4-cyclohexylphenyl)amino]-2-(1,3-oxazol-5-yl)-6-(propan-2-yl)-5,6-dihydro-7H-pyrrolo[3,4-d]pyrimidin-7-one;
4-[(4-cyclohexylphenyl)amino]-6-(propan-2-yl)-2-(1,3-thiazol-5-yl)-5,6-dihydro-7H-pyrrolo[3,4-d]pyrimidin-7-one;
2-(3,6-dihydro-2H-pyran-4-yl)-6-(propan-2-yl)-4-{[4-(propan-2-yl)phenyl]amino}-5,6-dihydro-7H-pyrrolo[3,4-d]pyrimidin-7-one;
4-{[4-(4-fluorophenoxy)phenyl]amino}-2-[(2R)-2-methylmorpholin-4-yl]-6-(propan-2-yl)-5,6-dihydro-7H-pyrrolo[3,4-d]pyrimidin-7-one;
2-(2-cyclopropylmorpholin-4-yl)-4-({4′-[(hept-6-yn-1-yl)oxy][1,1′-biphenyl]-4-yl}amino)-6-(propan-2-yl)-5,6-dihydro-7H-pyrrolo[3,4-d]pyrimidin-7-one;
2-(2-cyclopropylmorpholin-4-yl)-4-{[4′-(heptyloxy)[1,1′-biphenyl]-4-yl]amino}-6-(propan-2-yl)-5,6-dihydro-7H-pyrrolo[3,4-d]pyrimidin-7-one;
4-[(4′-{2-[3-(but-3-yn-1-yl)-3H-diaziren-3-yl]ethoxy}[1,1′-biphenyl]-4-yl)amino]-2-(2-cyclopropyl-morpholin-4-yl)-6-(propan-2-yl)-5,6-dihydro-7H-pyrrolo[3,4-d]pyrimidin-7-one;
2-[(2R)-2-methylmorpholin-4-yl]-4-[(4-pentylphenyl)amino]-6-(propan-2-yl)-5,6-dihydro-7H-pyrrolo[3,4-d]pyrimidin-7-one;
4-{[4-(butan-2-yl)phenyl]amino}-2-[(2R)-2-methylmorpholin-4-yl]-6-(propan-2-yl)-5,6-dihydro-7H-pyrrolo[3,4-d]pyrimidin-7-one;
4-{[4-(benzyloxy)phenyl]amino}-2-[(2R)-2-methylmorpholin-4-yl]-6-(propan-2-yl)-5,6-dihydro-7H-pyrrolo[3,4-d]pyrimidin-7-one;
2-(2-cyclopropylmorpholin-4-yl)-4-{[4-(pentafluoroethyl)phenyl]amino}-6-(propan-2-yl)-5,6-dihydro-7H-pyrrolo[3,4-d]pyrimidin-7-one;
2-(2-cyclopropylmorpholin-4-yl)-6-(propan-2-yl)-4-[(4-propylphenyl)amino]-5,6-dihydro-7H-pyrrolo[3,4-d]pyrimidin-7-one;
2-[(2R)-2-methylmorpholin-4-yl]-6-(propan-2-yl)-4-[(4-propylphenyl)amino]-5,6-dihydro-7H-pyrrolo[3,4-d]pyrimidin-7-one;
2-[(2R)-2-methylmorpholin-4-yl]-4-{[4-(pentafluoroethyl)phenyl]amino}-6-(propan-2-yl)-5,6-dihydro-7H-pyrrolo[3,4-d]pyrimidin-7-one;
2-(2-cyclopropylmorpholin-4-yl)-6-(propan-2-yl)-4-({4-[(propan-2-yl)oxy]phenyl}amino)-5,6-dihydro-7H-pyrrolo[3,4-d]pyrimidin-7-one;
4-[(4-cyclobutylphenyl)amino]-2-(morpholin-4-yl)-6-(propan-2-yl)-5,6-dihydro-7H-pyrrolo[3,4-d]pyrimidin-7-one;
4-{[4-(cyclopentyloxy)phenyl]amino}-2-[(2R)-2-methylmorpholin-4-yl]-6-(propan-2-yl)-5,6-dihydro-7H-pyrrolo[3,4-d]pyrimidin-7-one;
2-[(2R)-2-methylmorpholin-4-yl]-6-(propan-2-yl)-4-{[4-(2,2,2-trifluoroethyl)phenyl]amino}-5,6-dihydro-7H-pyrrolo[3,4-d]pyrimidin-7-one;
tert-butyl {2-[(4′-{[2-(2-cyclopropylmorpholin-4-yl)-7-oxo-6-(propan-2-yl)-6,7-dihydro-5H-pyrrolo[3,4-d]pyrimidin-4-yl]amino}[1,1′-biphenyl]-4-yl)oxy]ethyl}carbamate;
6-ethyl-2-[(2R)-2-methylmorpholin-4-yl]-4-{[4-(propan-2-yl)phenyl]amino}-5,6-dihydro-7H-pyrrolo[3,4-d]pyrimidin-7-one;
4-[(4-cyclohexylphenyl)amino]-6-ethyl-2-[(2R)-2-methylmorpholin-4-yl]-5,6-dihydro-7H-pyrrolo[3,4-d]pyrimidin-7-one;
tert-butyl {2-[(4′-{[2-(morpholin-4-yl)-7-oxo-6-(propan-2-yl)-6,7-dihydro-5H-pyrrolo[3,4-d]pyrimidin-4-yl]amino}[1,1′-biphenyl]-4-yl)oxy]ethyl}carbamate;
4-[(4′-{2-[3-(but-3-yn-1-yl)-3H-diaziren-3-yl]ethoxy}[1,1′-biphenyl]-4-yl)amino]-2-(morpholin-4-yl)-6-(propan-2-yl)-5,6-dihydro-7H-pyrrolo[3,4-d]pyrimidin-7-one;
2-(morpholin-4-yl)-6-(propan-2-yl)-4-({4′-[(prop-2-yn-1-yl)oxy][1,1′-biphenyl]-4-yl}amino)-5,6-dihydro-7H-pyrrolo[3,4-d]pyrimidin-7-one;
4-[(4-cyclohexylphenyl)amino]-6-[3-(dimethylamino)propyl]-2-[(2R)-2-methylmorpholin-4-yl]-5,6-dihydro-7H-pyrrolo[3,4-d]pyrimidin-7-one;
4-[(4-cyclohexylphenyl)amino]-6-[2-(dimethylamino)ethyl]-2-[(2R)-2-methylmorpholin-4-yl]-5,6-dihydro-7H-pyrrolo[3,4-d]pyrimidin-7-one;
4-[(4-cyclobutylphenyl)amino]-6-[3-(dimethylamino)propyl]-2-[(2R)-2-methylmorpholin-4-yl]-5,6-dihydro-7H-pyrrolo[3,4-d]pyrimidin-7-one;
4-[(4-cyclobutylphenyl)amino]-6-[2-(dimethylamino)ethyl]-2-[(2R)-2-methylmorpholin-4-yl]-5,6-dihydro-7H-pyrrolo[3,4-d]pyrimidin-7-one;
2-(morpholin-4-yl)-4-{[4-(propan-2-yl)phenyl]amino}-6-(prop-2-yn-1-yl)-5,6-dihydro-7H-pyrrolo[3,4-d]pyrimidin-7-one;
4-[(4-cyclohexylphenyl)amino]-2-(oxan-4-yl)-6-(propan-2-yl)-5,6-dihydro-7H-pyrrolo[3,4-d]pyrimidin-7-one;
4-[(4-cyclohexylphenyl)amino]-2-(1H-imidazol-1-yl)-6-(propan-2-yl)-5,6-dihydro-7H-pyrrolo[3,4-d]pyrimidin-7-one;
2-(3,6-dihydro-2H-pyran-4-yl)-4-[(2′-methyl[1,1′-biphenyl]-4-yl)amino]-6-(propan-2-yl)-5,6-dihydro-7H-pyrrolo[3,4-d]pyrimidin-7-one;
4-[(4′-{2-[3-(but-3-yn-1-yl)-3H-diaziren-3-yl]ethoxy}[1,1′-biphenyl]-4-yl)amino]-2-(3,6-dihydro-2H-pyran-4-yl)-6-(propan-2-yl)-5,6-dihydro-7H-pyrrolo[3,4-d]pyrimidin-7-one;
2-(3,6-dihydro-2H-pyran-4-yl)-4-[(2-fluoro[1,1′-biphenyl]-4-yl)amino]-6-(propan-2-yl)-5,6-dihydro-7H-pyrrolo[3,4-d]pyrimidin-7-one;
2-(morpholin-4-yl)-4-{[4-(pentafluoroethyl)phenyl]amino}-6-(propan-2-yl)-5,6-dihydro-7H-pyrrolo[3,4-d]pyrimidin-7-one;
4-[(2-fluoro[1,1′-biphenyl]-4-yl)amino]-2-(morpholin-4-yl)-6-(propan-2-yl)-5,6-dihydro-7H-pyrrolo[3,4-d]pyrimidin-7-one;
4-[(3′,4′-dichloro[1,1′-biphenyl]-4-yl)amino]-2-(morpholin-4-yl)-6-(propan-2-yl)-5,6-dihydro-7H-pyrrolo[3,4-d]pyrimidin-7-one;
2-(morpholin-4-yl)-6-(propan-2-yl)-4-{[4-(propan-2-yl)phenyl]amino}-5,6-dihydro-7H-pyrrolo[3,4-d]pyrimidin-7-one;
4-[(4-tert-butylphenyl)amino]-2-(morpholin-4-yl)-6-(propan-2-yl)-5,6-dihydro-7H-pyrrolo[3,4-d]pyrimidin-7-one;
4-[(2-methyl[1,1′-biphenyl]-4-yl)amino]-2-(morpholin-4-yl)-6-(propan-2-yl)-5,6-dihydro-7H-pyrrolo[3,4-d]pyrimidin-7-one;
4-[(4′-chloro[1,1′-biphenyl]-4-yl)amino]-2-(morpholin-4-yl)-6-(propan-2-yl)-5,6-dihydro-7H-pyrrolo[3,4-d]pyrimidin-7-one;
N-(4-cyclohexylphenyl)-2-[(2R)-2-methylmorpholin-4-yl]-5,7-dihydrofuro[3,4-d]pyrimidin-4-amine;
N-(4-cyclobutylphenyl)-2-(3,6-dihydro-2H-pyran-4-yl)-5,7-dihydrofuro[3,4-d]pyrimidin-4-amine;
N-(4-cyclohexylphenyl)-2-(2-cyclopropylmorpholin-4-yl)-5,7-dihydrofuro[3,4-d]pyrimidin-4-amine;
2-(2-cyclopropylmorpholin-4-yl)-N-[4′-(heptyloxy)[1,1′-biphenyl]-4-yl]-5,7-dihydrofuro[3,4-d]pyrimidin-4-amine;
2-[(2R)-2-methylmorpholin-4-yl]-N-{4′-[(6,6,6-trifluorohexyl)oxy][1,1′-biphenyl]-4-yl}-5,7-dihydrofuro[3,4-d]pyrimidin-4-amine;
N-(4-cyclohexylphenyl)-2-(2-methylpyridin-4-yl)-5,7-dihydrofuro[3,4-d]pyrimidin-4-amine;
N-(4-cyclohexylphenyl)-2-[(2R)-2-methylmorpholin-4-yl]pyrido[2,3-d]pyrimidin-4-amine (Compound D);
6-bromo-N-(4-cyclohexylphenyl)-2-[(2R)-2-methylmorpholin-4-yl]pyrido[2,3-d]pyrimidin-4-amine;
N-(4-cyclohexylphenyl)-2-(3,6-dihydro-2H-pyran-4-yl)pyrido[2,3-d]pyrimidin-4-amine;
N-(4-cyclohexylphenyl)-2-[(2R)-2-methylmorpholin-4-yl]-8-oxo-8lambda~5~-pyrido[2,3-d]pyrimidin-4-amine;
N-(4-cyclohexylphenyl)-6-ethyl-2-[(2R)-2-methylmorpholin-4-yl]pyrido[2,3-d]pyrimidin-4-amine;
4-[(4-cyclohexylphenyl)amino]-2-[(2R)-2-methylmorpholin-4-yl]pyrido[2,3-d]pyrimidine-6-carbonitrile;
methyl 4-[(4-cyclohexylphenyl)amino]-2-[(2R)-2-methylmorpholin-4-yl]pyrido[2,3-d]pyrimidine-6-carboxylate;
methyl (R)-4-((4-cyclohexylphenyl)amino)-2-(2-methylmorpholino)pyrido[2,3-d]pyrimidine-6-carboxylate (Compound E);
4-[(4-cyclohexylphenyl)amino]-2-[(2R)-2-methylmorpholin-4-yl]pyrido[2,3-d]pyrimidine-6-carboxylic acid;
4-[(4-cyclohexylphenyl)amino]-2-[(2R)-2-methylmorpholin-4-yl]pyrido[2,3-d]pyrimidine-6-carboxamide;
4-[(4-cyclohexylphenyl)amino]-2-(2-cyclopropylmorpholin-4-yl)pyrido[2,3-d]pyrimidine-6-carboxamide;
N-(4-cyclohexylphenyl)-6-methoxy-2-[(2R)-2-methylmorpholin-4-yl]pyrido[2,3-d]pyrimidin-4-amine;
(R)-N-(4-cyclohexylphenyl)-6-methoxy-2-(2-methylmorpholino)pyrido[2,3-d]pyrimidin-4-amine (Compound F);
N-{4-[(4-cyclohexylphenyl)amino]-2-[(2R)-2-methylmorpholin-4-yl]pyrido[2,3-d]pyrimidin-6-yl}acetamide;
N-4-(4-cyclohexylphenyl)-2-[(2R)-2-methylmorpholin-4-yl]pyrido[2,3-d]pyrimidine-4,6-diamine;
N-(4-cyclohexylphenyl)-2-(morpholin-4-yl)-6-(propan-2-yl)-6,7-dihydro-5H-pyrrolo[3,4-d]pyrimidin-4-amine;
N-(4-cyclohexylphenyl)-2-[(2R)-2-methylmorpholin-4-yl]-6-(propan-2-yl)-6,7-dihydro-5H-pyrrolo[3,4-d]pyrimidin-4-amine;
2-{4-[(4-cyclohexylphenyl)amino]-2-(3,6-dihydro-2H-pyran-4-yl)-5,7-dihydro-6H-pyrrolo[3,4-d]pyrimidin-6-yl}-N,N-dimethylacetamide (Compound G);
N-(4-cyclohexylphenyl)-2-(2-cyclopropylmorpholin-4-yl)-6-(1-methylpiperidin-4-yl)-6,7-dihydro-5H-pyrrolo[3,4-d]pyrimidin-4-amine;
2-(morpholin-4-yl)-6-(propan-2-yl)-4-{[4-(thiophen-2-yl)phenyl]amino}-5,6-dihydro-7H-pyrrolo[3,4-d]pyrimidin-7-one;
2-(morpholin-4-yl)-6-(propan-2-yl)-4-{[4-(thiophen-3-yl)phenyl]amino}-5,6-dihydro-7H-pyrrolo[3,4-d]pyrimidin-7-one;

and pharmaceutically acceptable salts thereof.

In embodiments, the compound according to Formula (I) is

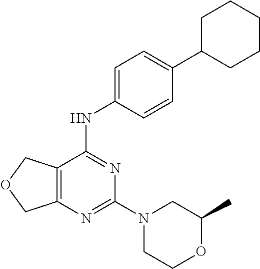 [SMILES: C[C@@H]1CN(c2nc3c(c(Nc4ccc(C5CCCCC5)cc4)n2)COC3)CCO1]

known as (R)-N-(4-cyclohexylphenyl)-2-(2-methylmorpholino)-5,7-dihydrofuro[3,4-d]pyrimidin-4-amine (also referred herein to as N-(4-cyclohexylphenyl)-2-[(2R)-2-methylmorpholin-4-yl]-5,7-dihydrofuro[3,4-d]pyrimidin-4-amine, or also referred to herein as Compound A or CmpA), or a pharmaceutically acceptable salt thereof.

In embodiments, the compound according to Formula (I) is Compound B:

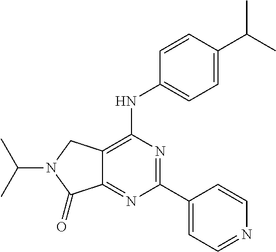 [SMILES: CC(C)c1ccc(Nc2nc(-c3ccncc3)nc3c2CN(C(C)C)C3=O)cc1]

### 6-isopropyl-4-((4-isopropylphenyl)amino)-2-(pyridin-4-yl)-5,6-dihydro-7H-pyrrolo[3,4-d]pyrimidin-7-one Compound B

In embodiments, the compound according to Formula (I) is Compound C:

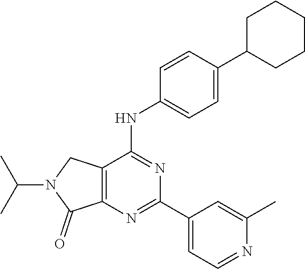 [SMILES: Cc1cc(-c2nc(Nc3ccc(C4CCCCC4)cc3)c3c(n2)C(=O)N(C(C)C)C3)ccn1]

### 4-[(4-cyclohexylphenyl)amino]-2-(2-methylpyridin-4-yl)-6-(propan-2-yl)-5,6-dihydro-7H-pyrrolo[3,4-d]pyrimidin-7-one Compound C

In embodiments, the compound according to Formula (I) is Compound D:

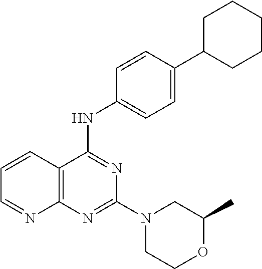 [SMILES: C[C@@H]1CN(c2nc(Nc3ccc(C4CCCCC4)cc3)c3cccnc3n2)CCO1]

### N-(4-cyclohexylphenyl)-2-[(2R)-2-methylmorpholin-4-yl]pyrido[2,3-d]pyrimidin-4-amine Compound D

In embodiments, the compound according to Formula (I) is Compound E:

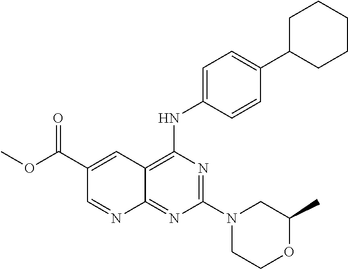 [SMILES: COC(=O)c1cnc2nc(N3CCO[C@H](C)C3)nc(Nc3ccc(C4CCCCC4)cc3)c2c1]

### methyl (R)-4-((4-cyclohexylphenyl)amino)-2-(2-methylmorpholino)pyrido[2,3-d]pyrimidine-6-carboxylate Compound E

In embodiments, the compound according to Formula (I) is Compound F:

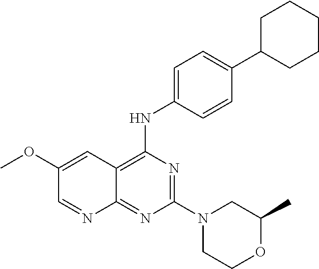 [SMILES: COc1cnc2nc(N3CCO[C@H](C)C3)nc(Nc3ccc(C4CCCCC4)cc3)c2c1]

### (R)-N-(4-cyclohexylphenyl)-6-methoxy-2-(2-methylmorpholino)pyrido[2,3-d]pyrimidin-4-amine Compound F

In embodiments, the compound according to Formula (I) is Compound G:

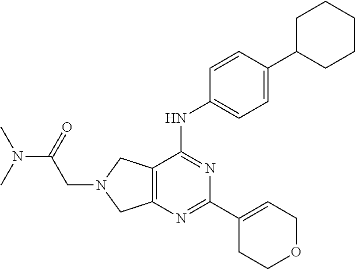 [SMILES: CN(C)C(=O)CN1Cc2nc(C3=CCOCC3)nc(Nc3ccc(C4CCCCC4)cc3)c2C1]

### 2-(4-((4-cyclohexylphenyl)amino)-2-(3,6-dihydro-2H-pyran-4-yl)-5,7-dihydro-6H-pyrrolo[3,4-d]pyrimidin-6-yl)-N,N-dimethylacetamide Compound G

In embodiments, the compound according to Formula (I) is:

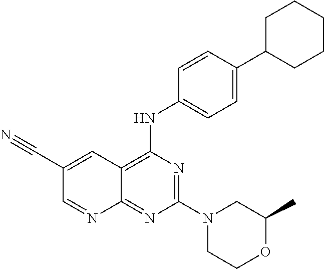 [SMILES: C[C@@H]1CN(c2nc(Nc3ccc(C4CCCCC4)cc3)c3cc(C#N)cnc3n2)CCO1]

known as (R)-4-((4-cyclohexylphenyl)amino)-2-(2-methylmorpholino)pyrido[2,3-d]pyrimidine-6-carbonitrile (also referred to as 4-[(4-cyclohexylphenyl)amino]-2-[(2R)-2-methylmorpholin-4-yl]pyrido[2,3-d]pyrimidine-6-carbonitrile or Compound H), or a pharmaceutically acceptable salt thereof.

Atoms of the compounds and salts described herein may exist as their isotopes. All compounds of Formula (I) where an atom is replaced by one or more of its isotopes (for example a compound according to Formula (I) where one or more carbon atom is an ¹¹C or ¹³C carbon isotope, or where one or more hydrogen atoms is a ²H or ³H isotope, or where one or more nitrogen atoms is a ¹⁵N isotope or where one of more oxygen atoms is an ¹⁷O or ¹⁸O isotope) are encompassed herein.

Compounds herein may exist in one or more geometrical, optical, enantiomeric, and diastereomeric forms, including, but not limited to, cis- and trans-forms, E- and Z-forms, and R-, S- and meso-forms. Unless otherwise stated a reference to a particular compound includes all such isomeric forms, including racemic and other mixtures thereof. Where appropriate such isomers can be separated from their mixtures by the application or adaptation of known methods (e.g., chromatographic techniques and recrystallisation techniques). Where appropriate such isomers can be prepared by the application or adaptation of known methods. In embodiments, a single stereoisomer is obtained by isolating it from a mixture of isomers (e.g., a racemate) using, for example, chiral chromatographic separation. In embodiments, a single stereoisomer is obtained through direct synthesis from, for example, a chiral starting material.

In embodiments, there is provided a compound according to Formula (I), or a pharmaceutically acceptable salt thereof, which is a single optical isomer being in an enantiomeric excess (% e.e.) of ≥95%, ≥98% or ≥99%. In embodiments, the single optical isomer is present in an enantiomeric excess (% e.e.) of ≥99%.

In embodiments, there is provided an N-oxide of a compound according to Formula (I) as herein defined, or a pharmaceutically acceptable salt thereof.

Compounds of Formula (I), where R⁷ is —NR¹⁰R¹¹ (i.e., R⁷ is linked by an aliphatic N atom), may for example be prepared as described in WO2021/180952 by the reaction of a compound according to Formula (V):

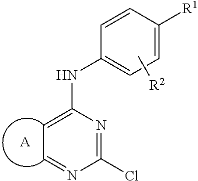

or a salt thereof, where R¹, R² and A are as defined herein, with an amine. The reaction is conveniently performed in a suitable solvent and at a suitable temperature, for example, di-isopropylethylamine in dimethylsulfoxide at a temperature of 20-100 °C, or TsOH in butanol at 80 °C.

When R⁷ is attached via a carbon atom, the compound according to Formula (I) can be made by the reaction of a compound according to Formula (V) with a boronic acid or ester of the Formula (VI), where R⁷ is as defined herein and each R is the same or different and represents —H, an aliphatic chain, or where together the two R groups form a ring with the boron and two oxygen atoms. The reaction is conveniently performed with a suitable base in the presence of a palladium catalyst and a solvent at a suitable temperature. For example, cesium carbonate or sodium carbonate and a palladium catalyst such as Pd(PPh₃)₄, in aqueous dioxane at a temperature in the range of 80-100 °C.

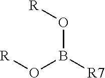

When R⁷ is linked via an aromatic N atom, the compound according to Formula (I) can be made by reaction of a compound according to Formula (V) with the anion of R⁷. For example, by reaction of the anion of imidazole, generated by treatment with a suitable base (for example, sodium hydride), in a suitable solvent (for example dimethylformamide), with a compound according to Formula (V).

A compound according to the Formula (V) may be prepared from a compound according to Formula (VII), or a salt thereof, where A is as defined herein, and a compound according to Formula (VIII), or a salt thereof, where R¹ and R² are as defined herein, in the presence of a base in a suitable solvent (for example, di-isopropylethylamine in tert butanol or dimethylsulfoxide) and at a suitable temperature (for example 20-100 °C).

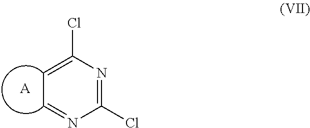

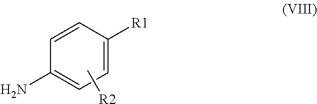

A compound according to Formula (I) may also be made in one pot from the reaction between a compound according to Formula (VII) with the stepwise addition of a compound according to Formula (VIII) and an amine R⁷. The reaction is conveniently performed in the presence of a base (for example, di-isopropylethylamine) in a suitable solvent (dimethylsulfoxide) at a suitable temperature (for example, a temperature of 20-100 °C).

A compound according to Formula (VII) may be made, for example, from a compound according to Formula (IX). Suitable conditions for this transformation are heating at a temperature of about 80 °C in POCl₃ in the presence of an amine base such as diethylphenylamine.

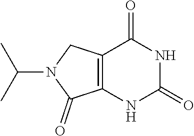

A compound according to the Formula (IX) may, for example, be prepared from a compound according to the Formula (X) by reaction with propan-2-amine and formaldehyde in a suitable solvent (for example, ethanol) at a suitable temperature (for example, a temperature of 0-80 °C).

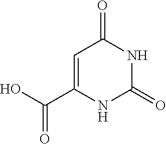 [SMILES: O=C(O)c1cc(=O)[nH]c(=O)[nH]1]

A compound according to the Formula (I), when R^{5a} and R^{5b} are both H, may also be made from reaction of a compound according to the Formula (XI), or a salt thereof, where R¹, R² and R⁷ are as defined herein, with a suitable amine, for example N,N-dimethylpropane-1,3-diamine. Suitable conditions for this reaction are HCl in ethanol at a temperature of about 190 °C in a sealed tube.

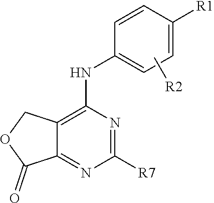

It will be appreciated that certain of the various ring substituents in the compounds herein may be introduced by standard aromatic substitution reactions or generated by conventional functional group modifications either prior to or immediately following the processes mentioned above. For example, compounds of Formula (I) may be converted into further compounds of Formula (I) by conventional functional group modifications. Such reactions and modifications include, for example, introduction of a substituent by means of an aromatic substitution reaction, C—H activation reaction, reduction of substituents, alkylation of substituents and oxidation of substituents. The reagents and reaction conditions for such procedures are well known in the chemical art. Particular examples of aromatic substitution reactions include the introduction of a halogen group.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

In embodiments, treatment of cancer is implemented by administering to a subject, e.g., a human, in need thereof, about 0.01 mg to about 1500 mg of a compound according to Formula (I), or a pharmaceutically acceptable salt thereof, such as Compound A, Compound B, Compound C, Compound D, Compound E, Compound F, Compound G, or Compound H, or a pharmaceutically acceptable salt of any of the foregoing. In embodiments, methods include treating cancer by administering to a subject, e.g., a human, in need thereof or diagnosed with cancer about 0.01 mg to about 1500 mg of a compound according to Formula (I), or a pharmaceutically acceptable salt thereof. In embodiments, the amount of a compound according to Formula (I), or a pharmaceutically acceptable salt thereof, such as Compound A, Compound B, Compound C, Compound D, Compound E, Compound F, Compound G, or Compound H, or a pharmaceutically acceptable salt of any of the foregoing, can be, e.g., between 0.1 and 1500 mg/day, or 0.01 mg/kg/day to 15 mg/kg/day, for treatment of cancer. For example, the daily dosage can be, e.g., in the range of about 0.01 to 1500 mg, 0.1 to 1250 mg, 0.1 to 1000 mg, 0.1 to 750 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 1 to 1500 mg, 1 to 1000 mg, 1 to 500 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 1500 mg, 5 to 1000 mg, 5 to 500 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 1500 mg, 10 to 1000 mg, 10 to 500 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 1500 mg, 15 to 1000 mg, 15 to 500 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 1500 mg, 20 to 1000 mg, 20 to 500 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 1500 mg, 25 to 1000 mg, 25 to 500 mg, 25 to 300 mg, 25 to 250 mg, 25 to 200 mg, 25 to 175 mg, 25 to 150 mg, 25 to 125 mg, 25 to 100 mg, 25 to 75 mg, 25 to 50 mg, 25 to 30 mg, 30 to 1500 mg, 30 to 1000 mg, 30 to 500 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 35 to 1500 mg, 35 to 1000 mg, 35 to 500 mg, 35 to 300 mg, 35 to 250 mg, 35 to 200 mg, 35 to 175 mg, 35 to 150 mg, 35 to 125 mg, 35 to 100 mg, 35 to 75 mg, 35 to 50 mg, 40 to 1500 mg, 40 to 1000 mg, 40 to 500 mg, 40 to 300 mg, 40 to 250 mg, 40 to 200 mg, 40 to 175 mg, 40 to 150 mg, 40 to 125 mg, 40 to 100 mg, 40 to 75 mg, 40 to 50 mg, 50 to 1500 mg, 50 to 1000 mg, 50 to 500 mg, 50 to 300 mg, 50 to 250 mg, 50 to 200 mg, 50 to 175 mg, 50 to 150 mg, 50 to 125 mg, 50 to 100 mg, 50 to 75 mg, 75 to 1500 mg, 75 to 1000 mg, 75 to 500 mg, 75 to 300 mg, 75 to 250 mg, 75 to 200 mg, 75 to 175 mg, 75 to 150 mg, 75 to 125 mg, 75 to 100 mg, 100 to 1500 mg, 100 to 1000 mg, 100 to 500 mg, 100 to 300 mg, 100 to 250 mg, 100 to 200 mg, 100 to 175 mg, 100 to 150 mg, 100 to 125 mg, 125 to 1500 mg, 125 to 1000 mg, 125 to 500 mg, 125 to 300 mg, 125 to 250 mg, 125 to 200 mg, 125 to 175 mg, 125 to 150 mg, 150 to 1500 mg, 150 to 1000 mg, 150 to 500 mg, 150 to 300 mg, 150 to 250 mg, 150 to 200 mg, 150 to 175 mg, 175 to 1500 mg, 175 to 1000 mg, 175 to 500 mg, 175 to 300 mg, 175 to 250 mg, 175 to 200 mg, 200 to 1500 mg, 200 to 1000 mg, 200 to 500 mg, 200 to 300 mg, 200 to 250 mg, 250 to 1500 mg, 250 to 1000 mg, 250 to 500 mg, 250 to 300 mg, 7.5 to 15 mg, 2.5 to 5 mg, 1 to 5 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 7.5 mg, 10 mg, 12.5 mg, 15 mg, 17.5 mg, 20 mg, 22.5 mg, 25 mg, 27.5 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, 400 mg and 500 mg being examples.

In embodiments, pharmaceutical compositions for treating cancer may include a compound according to Formula (I), or a pharmaceutically acceptable salt thereof, such as Compound A, Compound B, Compound C, Compound D, Compound E, Compound F, Compound G, or Compound H, or a pharmaceutically acceptable salt of any of the foregoing, in an amount of, e.g., about 0.01 to 1500 mg, 0.01 to 1250 mg, 0.01 to 1000 mg, 0.01 to 750 mg, 0.01 to 500 mg, 0.01 to 250 mg, 0.01 to 100 mg, 0.01 to 50 mg, 0.01 to 25 mg, 0.01 to 10 mg, 0.01 to 5 mg, 0.01 to 1 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.5 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 1 mg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.1 mg, 0.25 mg, 0.5 mg, 0.75 mg, 1 mg, 2.5 mg, 5 mg, 7.5 mg, 10 mg, 12.5 mg, 15 mg, 17.5 mg, 20 mg, 22.5 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples.

Typically, dosages of Formula (I), or a pharmaceutically acceptable salt thereof, may be administered to a subject diagnosed with cancer once, twice, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four times daily, every other day, once weekly, or once a month. In embodiments, a compound according to Formula (I), or a pharmaceutically acceptable salt thereof, is administered every hour. In embodiments, a compound according to Formula (I), or a pharmaceutically acceptable salt thereof, is administered to a subject diagnosed with cancer as a continuous infusion. In embodiments, a compound according to Formula (I), or a pharmaceutically acceptable salt thereof, is administered to a subject diagnosed with cancer once a day (e.g., morning or night), or twice a day, (e.g., morning and evening), three times a day (e.g., at breakfast, lunch, and dinner, or every 8 hours), four times a day (e.g., breakfast, lunch, dinner and at bedtime), or every hour at a dose of 0.01-1000 mg/administration.

In embodiments, a compound according to Formula (I), or a pharmaceutically acceptable salt thereof, such as Compound A, Compound B, Compound C, Compound D, Compound E, Compound F, Compound G, or Compound H, or a pharmaceutically acceptable salt of any of the foregoing, is administered to a subject diagnosed with cancer herein in an amount of 2000 mg/day, 1500 mg/per day, 1400 mg/per day, 1300 mg/per day, 1200 mg/per day, 1000 mg/per day, 900 mg/per day, 800 mg/per day, 700 mg/per day, 600 mg/per day, 500 mg/per day, 400 mg/per day, 300 mg/per day, 200 mg/per day, 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 3 mg/per day, 2 mg/per day, 1 mg/per day, in one or more doses. Dosages can be lower for infants and children than for adults. In embodiments, an infant or pediatric dose can be about 0.1 to 1500 mg per day once or in 2, 3, 4, 5, 6, 7, 8, 9, 10, 11, 12, 13, 14, 15, 16, 17, 18, 19, 20, 21, 22, 23, or 24 divided doses. In embodiments, a pediatric dose can be 0.05 mg/kg/day to 1500 mg/kg/day. In embodiments, the subject may be started at a low dose and the dosage is escalated over time. It should be understood that the above amounts are exemplary and doses of a compound according to Formula (I), or a pharmaceutically acceptable salt thereof, can include different amounts and varying ranges within a continuum between the minimum or maximum amounts described above in connection with a compound according to Formula (I), or a pharmaceutically acceptable salt thereof.

In embodiments, the step of administering results in at least one of the following: a 5% to 10% reduction in size of tumors, at least a 5% to 10% reduction in number of cancer metastases, or at least a 5% to 10% improvement in clinical signs and symptoms related to cancer. The term “administration” and grammatical variations thereof such as “administer” and “administering”, as used herein means providing a predetermined substance to an individual or a patient by any appropriate method. For example, a compound according to Formula (I), or a pharmaceutically acceptable salt thereof, may be administered orally or parenterally (for example, by applying in injectable formulations intravenously, subcutaneously, intraperitoneally, or topically). The dosage may vary depending on the patient's body weight, age, sex, health condition, diet, administration time, administration method, excretion rate, the severity of the disease and the like. Liquid formulations for oral administration of a compound according to Formula (I), or a pharmaceutically acceptable salt thereof, include suspensions, oral liquids, emulsions, syrups and the like. In addition to water and liquid paraffin which are simple diluents commonly used, various excipients such as wetting agents, sweeteners, flavors, preservatives and the like may be included. Formulations for parenteral administration include sterile aqueous solutions, non-aqueous solvents, suspensions, emulsions, freeze-dried formulations, suppositories, and the like.

In embodiments, a compound according to Formula (I), or a pharmaceutically acceptable salt thereof is administered to a subject having cancer via a pharmaceutical composition. Pharmaceutical compositions herein may be administered by any device capable of moving the active substance to target cells. Pharmaceutical compositions herein encompass dosage forms. Dosage forms herein encompass unit doses. In embodiments, as discussed below, various dosage forms including conventional release formulations and modified release formulations can be administered one or more times daily. In embodiments, a compound according to Formula (I), or a pharmaceutically acceptable salt thereof, is administered to a subject once or twice a day (e.g., morning and/or night), or three times a day (morning, afternoon and night) or four times a day (morning, afternoon, evening and night). Any suitable route of administration may be utilized, e.g., oral, rectal, nasal, pulmonary, vaginal, sublingual, transdermal, subcutaneous, intravenous, intraarterial, intramuscular, intraperitoneal, intratumoral, intracranial and intraventricular routes.

Suitable dosage forms include tablets, capsules, oral liquids, powders, aerosols, transdermal modalities such as topical liquids, patches, creams and ointments, parenteral formulations and suppositories. In embodiments, a compound according to Formula (I), or a pharmaceutically acceptable salt thereof, is used to manufacture a medicament for treatment of cancer or inhibiting cancer proliferation and metastasis. Those skilled in the art are familiar with pharmaceutical compounding techniques for formulating and manufacturing pharmaceutical compositions. See, e.g., E. W. Martin ed., Remington's Pharmaceutical Sciences, 18th ed. (1990), Mack Publishing Co.

In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in cancer symptoms for more than 1 hour after administration to the subject. In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in one or more cancer symptoms for more than 2 hours after administration to the subject. In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in one or more cancer symptoms for more than 3 hours after administration to the subject. In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in one or more cancer symptoms for more than 4 hours after administration to the subject. In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in one or more cancer symptoms for more than 6 hours after administration to the subject. In embodiments, methods of treating cancer including inhibiting cancer proliferation and metastasis are provided which include administering to a subject in need thereof a pharmaceutical composition including a compound according to Formula (I), or a pharmaceutically acceptable salt thereof, wherein the composition provides improvement in one or more cancer symptoms for more than 8, 10, 12, 14, 16, 18, 20, 22 or 24 hours after administration to the subject. In embodiments, the pharmaceutical compositions provide improvement of next day functioning of the subject. For example, the pharmaceutical compositions may provide improvement in one or more cancer symptoms for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

The compounds of Formula (I), or pharmaceutically acceptable salts thereof, may be administered as pharmaceutical compositions, including one or more pharmaceutically acceptable excipients. Therefore, in embodiments, there is provided a pharmaceutical composition including a compound of Formula (I), or a pharmaceutically acceptable salt thereof, and at least one pharmaceutically acceptable excipient. In embodiments, as mentioned previously, pharmaceutical compositions herein may be provided with conventional release or modified release profiles. Pharmaceutical compositions may be prepared using a pharmaceutically acceptable “carrier” or “excipient” composed of materials that are considered safe and effective. “Carrier” and “excipient” are used interchangeably herein. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers. In embodiments, pharmaceutical compositions herein may include a pharmaceutically acceptable additive, which is exemplified by starch, gelatinized starch, microcrystalline cellulose, milk sugar, povidone, colloidal silicon dioxide, calcium hydrogen phosphate, lactose, mannitol, Arabia rubber, pregelatinized starch, corn starch, cellulose powder, hydroxypropyl cellulose, Opadry, sodium starch glycolate, carnauba wax, synthetic aluminum silicate, stearic acid, magnesium stearate, aluminum stearate, calcium stearate, white sugar, dextrose, sorbitol, talc, etc. In embodiments, a pharmaceutically acceptable additive can be added to the composition in an amount of 0.1 to 90 parts by weight but is not limited thereto.

The pharmaceutical compositions may be in a form suitable for oral use (for example as tablets, lozenges, hard or soft capsules, films, dragees, aqueous or oily suspensions, emulsions, dispersible powders or granules, syrups or elixirs), for topical use (for example as creams, ointments, gels, lotions, transdermal patches, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution or suspension for intravenous, subcutaneous or intramuscular dosing), or as a suppository for rectal dosing. The compositions may be obtained by conventional procedures using conventional pharmaceutical excipients well known in the art. Thus, compositions intended for oral use may contain, for example, one or more coloring, sweetening, flavoring and/or preservative agents.

In embodiments, a pharmaceutical composition herein may contain a compound according to Formula (I) in any of the amounts set forth herein and a diluent in an amount from 30% to 90%. Examples of diluents include lactose, microcrystalline cellulose, starch, calcium phosphate, calcium carbonate, sucrose, mannitol, maltodextrin and sorbitol. In embodiments, a pharmaceutical composition herein may contain a compound according to Formula (I) and a lubricant in an amount, e.g., from 0.25% to 5.0%. Examples of lubricants include magnesium stearate, stearic acid, sodium stearyl fumarate, talc, polyethylene glycols and silicon dioxide. Examples of water-soluble lubricants include sodium benzoate, polyethylene glycol, and adipic acid. In embodiments, a pharmaceutical composition herein may contain a compound according to Formula (I) and a disintegrant in an amount, e.g., from 1.0% to 10.0%. There are two classes of disintegrants: traditional disintegrants, such as starch, and super disintegrants, which include croscarmellose sodium, crospovidone, and sodium starch glycolate.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. Super disintegrants may form a majority of the dosage form. This mode of administration can be beneficial to people who may have problems swallowing tablets whether it be from physical infirmity or psychiatric in nature. Subjects with addiction or insomnia may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which a compound according to Formula (I), or a pharmaceutically acceptable salt thereof, is applied to beads, e.g., confectioners nonpareil beads, and then coated with conventional release retarding materials such as waxes, enteric coatings and the like. In embodiments, beads can be formed in which a compound according to Formula (I), or a pharmaceutically acceptable salt thereof, is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as a compound according to Formula (I), or a pharmaceutically acceptable salt thereof, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles is covered with a coating which delays release of the drug.

Delayed release dosage forms are known to those skilled in the art. For example, coated delayed release beads or granules in which a compound according to Formula (I), or a pharmaceutically acceptable salt thereof, is applied to beads, e.g., confectioners nonpareil beads, and then coated with conventional release delaying materials such as waxes, enteric coatings and the like. In embodiments, beads can be formed in which a compound according to Formula (I), or a pharmaceutically acceptable salt thereof, is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of a compound according to Formula (I), or a pharmaceutically acceptable salt thereof, can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, a compound according to Formula (I), or a pharmaceutically acceptable salt thereof, is incorporated into porous inert carriers that provide delayed release profiles. In embodiments, the porous inert carriers incorporate channels or passages from which the drug diffuses into surrounding fluids. In embodiments, a compound according to Formula (I), or a pharmaceutically acceptable salt thereof, is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion or implantation into the body and may be packaged in either single-dose or multi-dose containers. In embodiments, liquid pharmaceutical compositions for parenteral administration to a subject include a compound according to Formula (I), or a pharmaceutically acceptable salt thereof, in any of the respective amounts described above. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, pharmaceutical compositions for parenteral administration include respective amounts described above for a compound according to Formula (I), or a pharmaceutically acceptable salt thereof. In embodiments, pharmaceutical compositions for parenteral administration include about 0.05 mg to about 500 mg a compound according to Formula (I), or a pharmaceutically acceptable salt thereof. In embodiments, pharmaceutical compositions for parenteral administration to a subject include a compound according to Formula (I), or a pharmaceutically acceptable salt thereof, at a respective concentration of about 0.005 mg/ml to about 500 mg/ml. In embodiments, the pharmaceutical composition for parenteral administration includes a compound according to Formula (I), or a pharmaceutically acceptable salt thereof, at a respective concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes a compound according to Formula (I), or a pharmaceutically acceptable salt thereof, at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for parenteral administration is provided wherein the pharmaceutical composition is stable for at least six months. In embodiments, the pharmaceutical compositions for parenteral administration exhibit no more than about 5% decrease in a compound according to Formula (I), or a pharmaceutically acceptable salt thereof, e.g., 3 months or 6 months. In embodiments, the amount of a compound according to a compound according to Formula (I), or a pharmaceutically acceptable salt thereof, degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a subject in need thereof.

The pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of a compound according to Formula (I), or a pharmaceutically acceptable salt thereof, used in the composition. Thus, parenteral compositions are provided wherein there is no incompatibility between any of the components of the dosage form.

In embodiments, parenteral compositions including a compound according to Formula (I), or a pharmaceutically acceptable salt thereof, include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions including a compound according to Formula (I), or a pharmaceutically acceptable salt thereof, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions may be administered as needed, e.g., once, twice, thrice or four or more times daily, or continuously depending on the subject's needs.

In embodiments, parenteral compositions of a compound according to Formula (I), or a pharmaceutically acceptable salt thereof, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

It should be understood that respective dosage amounts of a compound according to Formula (I), or a pharmaceutically acceptable salt thereof, that are provided herein are applicable to all the dosage forms described herein including conventional dosage forms, modified dosage forms, as well as the parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

In embodiments, provided herein are methods of treating cancer including administering to a subject in need thereof or diagnosed with cancer a pharmaceutical composition including a compound according to Formula (I), or a pharmaceutically acceptable salt thereof, in a respective amount described herein, wherein the composition provides an in vivo plasma profile of a compound according to Formula (I) having a Cₘₐₓ less than about 800 ng/ml. In embodiments, the composition provides improvement for more than 6 hours after administration to the subject.

In embodiments, pharmaceutical compositions including a compound according to Formula (I), or a pharmaceutically acceptable salt thereof, provide an in vivo plasma profile of a compound according to Formula (I) having a Cₘₐₓ less than about, e.g., 2000 ng/ml, 1000 ng/ml, 850 ng/ml, 800 ng/ml, 750 ng/ml, 700 ng/ml, 650 ng/ml, 600 ng/ml, 550 ng/ml, 450 ng/ml, 400 ng/ml 350 ng/ml, or 300 ng/ml. In embodiments, the pharmaceutical composition provides an in vivo plasma profile of a compound according to Formula (I), or a pharmaceutically acceptable salt thereof, having a Cₘₐₓ less than about, e.g., 250 ng/ml, 200 ng/ml 150 ng/ml, or 100 ng/ml. In embodiments, for example, in the case of Compound A, the Cₘₐₓ is rapidly achieved within about 10 minutes of IV infusion. In embodiments, the pharmaceutical composition provides improvement in one or more cancer symptoms for more than 6 hours after administration.

In embodiments, provided herein are methods of treating cancer including administering to a subject in need thereof a pharmaceutical composition containing a compound according to Formula (I), or a pharmaceutically acceptable salt thereof, wherein the composition provides a consistent in vivo plasma profile having a AUC_{0-∞} of less than about 900 ng·hr/ml. In embodiments, the compositions provide an in vivo plasma profile of a compound according to Formula (I), or a pharmaceutically acceptable salt thereof, having a AUC_{0-∞} of less than about, e.g., 850 ng·hr/ml, 800 ng·hr/ml, 750 ng·hr/ml, or 700 ng·hr/ml.

In embodiments, provided herein are methods of treating cancer including administering to a subject in need thereof a pharmaceutical composition comprising a compound according to Formula (I), or a pharmaceutically acceptable salt thereof, wherein the pharmaceutical composition provides an in vivo plasma profile of a compound according to Formula (I), or a pharmaceutically acceptable salt thereof, having a AUC_{0-∞} of less than about, e.g., 650 ng·hr/ml, 600 ng·hr/ml, 550 ng·hr/ml, 500 ng·hr/ml, or 450 ng·hr/ml. In embodiments, the composition provides an in vivo plasma profile of a compound according to Formula (I), or a pharmaceutically acceptable salt thereof, having a AUC_{0-∞} of less than about, e.g., 400 ng·hr/ml, 350 ng·hr/ml, 300 ng·hr/ml, 250 ng·hr/ml, or 200 ng·hr/ml. In embodiments, the pharmaceutical composition provides an in vivo plasma profile of a compound according to Formula (I), or a pharmaceutically acceptable salt thereof, having a AUC_{0-∞} of less than about, e.g., 150 ng·hr/ml, 100 ng·hr/ml, 75 ng·hr/ml, or 50 ng·hr/ml.

In embodiments, the Tₘₐₓ provided by a pharmaceutical composition containing a compound according to Formula (I), or a pharmaceutically acceptable salt thereof, is less than 3 hours. In embodiments, the Tₘₐₓ provided by the pharmaceutical composition containing a compound according to Formula (I), or a pharmaceutically acceptable salt thereof, is less than 2.5 hours. In embodiments, the Tₘₐₓ provided by the pharmaceutical composition containing a compound according to Formula (I), or a pharmaceutically acceptable salt thereof, is less than 2 hours. In embodiments, the Tₘₐₓ provided by the pharmaceutical composition containing a compound according to Formula (I), or a pharmaceutically acceptable salt thereof, is less than 1.5 hours. In embodiments, the Tₘₐₓ provided by the pharmaceutical composition containing a compound according to Formula (I), or a pharmaceutically acceptable salt thereof, is less than 1 hour. In embodiments, the Tₘₐₓ provided by the pharmaceutical composition containing a compound according to Formula (I), or a pharmaceutically acceptable salt thereof, is less than 0.5 hour. In embodiments, the Tₘₐₓ provided by the pharmaceutical composition containing a compound according to Formula (I), or a pharmaceutically acceptable salt thereof, is less than 0.25 hour. For example, the Tₘₐₓ of Compound A after IV administration is about 10 minutes.

In embodiments, the pharmaceutical composition containing a compound according to Formula (I), or a pharmaceutically acceptable salt thereof, provides a dissolution of at least about 80% within the first 20 minutes of administration to a subject in need thereof. In embodiments, the pharmaceutical composition containing a compound according to Formula (I), or a pharmaceutically acceptable salt thereof, provides a dissolution of at least about, e.g., 85%, 90% or 95% within the first 20 minutes of administration to a subject in need thereof. In embodiments, the pharmaceutical composition containing a compound according to Formula (I), or a pharmaceutically acceptable salt thereof, provides a dissolution of at least 80% within the first 10 minutes of administration to a subject in need thereof.

In embodiments, a method of decreasing activity of BCRP is provided which includes contacting cells expressing BCRP with a compound according to Formula (I), or a pharmaceutically acceptable salt thereof. In embodiments, use of a compound according to Formula (I), or a pharmaceutically acceptable salt thereof, for decreasing activity of BCRP is provided which includes contacting cells expressing BCRP with the compound.

In embodiments, the cells are in vivo. In embodiments, the cells are in vitro. In embodiments, the cells are human cells. In embodiments, the cells are human cells from a patient. In embodiments, the cells are animal cells. In embodiments, the cells are non-human mammalian cells. In embodiments, the cells are primate cells. In embodiments, the cells are canine cells. In embodiments, the cells are porcine cells. In embodiments, the cells are rat cells. In embodiments, the cells are mouse cells. In embodiments, the cells are BCRP-overexpressing cells. In embodiments, the cells are intestinal enterocytes, hepatocytes, kidney proximal tubule cells, brain endothelial cells, placental cells, stem cells, mammary gland cells, breast epithelial or blood vessel cells.

The term “resistant to a chemotherapeutic agent” or “resistance to a chemotherapeutic agent” as used herein defines a reduction or loss of therapeutic effectiveness of a chemotherapeutic agent in the treatment of a cancer condition. The resistance of a cancer to a chemotherapeutic agent can be due to BCRP, e.g. to expression or over-expression of BCRP. Even low expression of BCRP can be responsible for the cancer resistance to a chemotherapeutic agent. “Over-expressing” or “over-expression” means that the level of expression of BCRP or of its mRNA is higher, e.g. 1.25, 1.5, 2, 4, 6, 10, 20 or more times higher, than the corresponding level of the BCRP in a healthy patient or in corresponding normal tissues not harboring cancer or in cancer that is not resistant to a chemotherapeutic agent.

Combination therapies utilizing a compound according to Formula (I), or a pharmaceutically acceptable salt thereof, can include administration of the active agents together in the same admixture, or in separate admixtures. In embodiments, the pharmaceutical composition can include two, three, or more active agents. In embodiments, combination therapies involve administration of a compound according to Formula (I), or a pharmaceutically acceptable salt thereof, in combination with other active agents. A compound according to Formula (I), or a pharmaceutically acceptable salt thereof, and other active agents may be administered together at the same time or separately in spaced apart intervals. A compound according to Formula (I), or a pharmaceutically acceptable salt thereof, and the other active agents may be administered in a single dosage form or in separate dosage forms. A compound according to Formula (I), or a pharmaceutically acceptable salt thereof, and other active agents may be administered simultaneously or in spaced-apart intervals to the subject via separate routes of administration.

In embodiments, a therapeutically effective amount of a chemotherapeutic agent is administered in combination with a compound according to Formula (I), or a pharmaceutically acceptable salt thereof, e.g., Compound A, Compound B, Compound C, Compound D, Compound E, Compound F, Compound G, or Compound H. Therapeutically effective amounts of chemotherapeutic agent are known or otherwise routinely determined by those skilled in the art.

Co-administration of one or more chemotherapeutic agents and a compound according to Formula (I), or a pharmaceutically acceptable salt thereof, e.g., Compound A, Compound B, Compound C, Compound D, Compound E, Compound F, Compound G, or Compound H, may be sequential. In embodiments, one or more chemotherapeutic agents may be administered to the subject after administering a compound according to Formula (I), or a pharmaceutically acceptable salt thereof, e.g., Compound A, Compound B, Compound C, Compound D, Compound E, Compound F, Compound G, or Compound H, to the subject. In embodiments, one or more chemotherapeutic agents may be administered to the subject prior to administering a compound according to Formula (I), or a pharmaceutically acceptable salt thereof, e.g., Compound A, Compound B, Compound C, Compound D, Compound E, Compound F, Compound G, or Compound H, to the subject. Alternatively, one or more chemotherapeutic agents and a compound according to Formula (I), or a pharmaceutically acceptable salt thereof, e.g., Compound A, Compound B, Compound C, Compound D, Compound E, Compound F, Compound G, or Compound H, may be administered simultaneously. In embodiments, bolus IV administration before chemotherapeutic agent infusion may help maximize intracellular drug retention for short-duration agents like paclitaxel or irinotecan.

In embodiments, the combinations result in a more than an additive effect on the treatment of cancer. Thus, treatment is provided for cancer with a combination of agents that combined, may provide a synergistic effect that enhances efficacy and reduces side effects. In embodiments, a compound according to Formula (I), or a pharmaceutically acceptable salt thereof, e.g., Compound A, Compound B, Compound C, Compound D, Compound E, Compound F, Compound G, or Compound H, is administered in combination with chemotherapeutic agents. As mentioned above, a compound according to Formula (I), or a pharmaceutically acceptable salt thereof, inhibits activity of breast cancer resistance protein (BCRP). For example, Compound A is a highly selective BCRP inhibitor that demonstrates superior potency, specificity, and a favorable safety profile compared to existing inhibitors.

Compound A exhibits High Selectivity. Unlike many broad-spectrum ABC transporter inhibitors, Compound A exhibits minimal off-target effects on P-glycoprotein (P-gp) and multidrug resistance-associated proteins (RPs), reducing toxicity concerns. Compound A exhibits Increased Potency. The half-maximal inhibitory concentration (IC₅₀) of Compound A against BCRP is significantly lower than that of standard inhibitors such as Ko143 and Fumitremorgin C (FTC). Compared to FTC, which has been associated with neurotoxicity, Compound A has demonstrated a safer profile in preclinical studies. Specific cancer types where Compound A may have a strong impact include: Acute Myeloid Leukemia (AML): High BCRP expression has been correlated with poor clinical outcomes and resistance to mitoxantrone and topotecan. Non-Small Cell Lung Cancer (NSCLC): BCRP inhibition has been linked to improved responses to platinum-based chemotherapy. Breast Cancer: Compound A can enhance the retention of anthracyclines and taxanes in resistant breast cancer cells. Gastrointestinal Tumors: Given the role of BCRP in the efflux of irinotecan and methotrexate, Compound A can improve drug retention in colorectal and gastric cancers.

BCRP inhibition by a compound according to Formula (I), or a pharmaceutically acceptable salt thereof, e.g., Compound A, Compound B, Compound C, Compound D, Compound E, Compound F, Compound G, or Compound H, enhances radiotherapy and photodynamic therapy (PDT) by preventing or reducing the active efflux of these chemotherapeutic agents, thereby increasing their intracellular retention and efficacy. Current dosing regimens for radiopharmaceuticals and photosensitizers often require high initial doses to compensate for rapid efflux in BCRP-expressing tumors. This approach increases systemic exposure and toxicity, limiting dose escalation in patients who need higher drug concentrations.

Radiotherapy involves radiopharmaceuticals which contain a radioactive substance that are used in both diagnosis and treatment of diseases, including cancer. They are radioactive compounds used in nuclear medicine. Radiopharmaceuticals are made of two parts: A radioisotope (the radioactive part) and a carrier molecule (which targets specific tissues or cells). Therapeutic radiopharmaceuticals are designed to kill cancer cells. The radiopharmaceutical seeks out and binds to cancer cells, delivering radiation directly. The radioisotope emits radiation (e.g., alpha or beta particles) that damages DNA causing the cancer cells to die. Radiopharmaceuticals are often used for: thyroid cancer (e.g., Iodine-131 targets thyroid tissue); prostate cancer (e.g., Lutetium-177 PSMA therapy); neuroendocrine tumors (e.g., Lutetium-177 Dotatate); Actinium-225 PSMA: used for advanced prostate cancer; Radium-223 (Xofigo): used for prostate cancer with bone metastases; Yttrium-90 (Y-90) microspheres: used for liver cancer. Other radiopharmaceuticals are ⁹⁰Y-Ibritumomab Tiuxetan (Zevalin), ¹³¹I-Tositumomab (Bexxar), and ¹⁷⁷Lu-DOTATATE (Lutathera) to name a few. Radiopharmaceuticals are highly targeted, so there are fewer side effects compared to traditional chemo or external radiation. They can reach tumors that are difficult to operate on or treat with other methods. They can be used alongside other treatments (e.g., chemotherapy or immunotherapy). However, many radiopharmaceuticals are cleared quickly from the bloodstream, leading to low tumor uptake and requiring high systemic doses to achieve therapeutic effects.

Compounds according to Formula (I), or a pharmaceutically acceptable salt thereof, e.g., Compound A, Compound B, Compound C, Compound D, Compound E, Compound F, Compound G, or Compound H, enhance intracellular retention of radiopharmaceuticals via BCRP inhibition. Accordingly, co-administration of compounds according to Formula (I), or a pharmaceutically acceptable salt thereof, e.g., Compound A, Compound B, Compound C, Compound D, Compound E, Compound F, Compound G, or Compound H, with radiopharmaceuticals, maximizes tumor exposure and lowers radiation dose requirements. In addition, co-administration of a compound according to Formula (I), or a pharmaceutically acceptable salt thereof, e.g., Compound A, Compound B, Compound C, Compound D, Compound E, Compound F, Compound G, or Compound H, allows for lower starting doses while maintaining therapeutic effectiveness by increasing tumor retention times. This would reduce bone marrow toxicity and off-target effects, particularly in hematologic malignancies.

Compound A is characterized by rapid clearance following IV administration, with a Tₘₐₓ of 0.167 hours (10 minutes post-infusion) in both rats and dogs. This means that Compound A reaches peak plasma concentration within 10 minutes but is also cleared quickly. In embodiments, repeat or continuous infusion for sustained BCRP inhibition may be advantageous. In embodiments, Compound A can be administered 10-15 minutes before radiopharmaceuticals to ensure peak inhibition at the time of infusion. Repeat infusion of Compound A every 4-6 hours may be needed for radiopharmaceuticals with longer systemic half-lives (e.g., Lutathera).

Photodynamic therapy is a medical treatment that relies on photosensitizers that accumulate in cancer cells and generate reactive oxygen species (ROS) upon light activation, leading to tumor cell death. PDT uses a combination of a photosensitizing agent that becomes active when exposed to light, a specific wavelength of light, and oxygen in the tissue. The photosensitizer is either applied to the skin or injected into the bloodstream, and it accumulates in abnormal or cancerous cells more than in healthy cells. However, BCRP-mediated efflux significantly reduces intracellular concentrations of these photosensitizers, weakening PDT efficacy, particularly in resistant tumors. Many PDT agents require high doses due to rapid efflux, leading to increased risk of systemic phototoxicity.

The ability of compounds according to Formula (I), or a pharmaceutically acceptable salt thereof, e.g., Compound A, Compound B, Compound C, Compound D, Compound E, Compound F, Compound G, or Compound H, to enhance intracellular retention of photosensitizers via BCRP inhibition increases efficacy and reduces required light exposure—a significant advantage in clinical settings where phototoxicity and drug clearance limit effectiveness. Higher intracellular retention via compounds according to Formula (I), or a pharmaceutically acceptable salt thereof, e.g., Compound A, Compound B, Compound C, Compound D, Compound E, Compound F, Compound G, or Compound H, would improve treatment responses at lower light doses, reducing collateral damage to surrounding healthy tissues. Additionally, it can enable PDT in tumors previously deemed resistant due to rapid drug efflux. In embodiments, Compound A can be administered 15-30 minutes before PDT agent infusion to allow optimal photosensitizer retention. Given Compound A's short half-life, a second dose may be needed if PDT light activation is delayed beyond 2-3 hours. Light exposure in PDT may be reduced by up to 30-50% upon coadministration with Compound A due to increases in intracellular photosensitizer concentrations.

PDT agents include aminolevulinic acid (ALA) (Levulan), Silicon Phthalocyanine Pc 4, m-tetrahydroxyphenylchlorin (mTHPC) and mono-L-aspartyl chlorin e6 (NPe6). Photosensitizers commercially available for clinical use include, porfimer sodium (Photofrin): esophageal cancer, non-small cell lung cancer, and Barrett's esophagus (red light (~630 nm)), verteporfin (Visudyne): basal cell carcinoma (Blue light (~417 nm) or red light), temoporfin (Foscan): Advanced head and neck cancers (Red light (~652 nm)), methyl aminolevulinate (Metvix): actinic keratosis, basal cell carcinoma, Bowen's disease (Red light (~630 nm)), hexylaminolevulinate (Hexvix or Cysview): bladder cancer (Blue light (~375-440 nm)), Pheophorbide A (Pha), Protoporphyrin IX (PpIX), Benzoporphyrin Derivatives (BPDs), talaporfin sodium (Laserphyrin), motexafin lutetium (Antrin), HPPH (Photochlor), sulfonated aluminium phthalocyanines (Photosens), rostaporfin (Photrex), BF-200 ALA, fimaporfin (Amphinex) and azadipyrromethenes.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of cancer measured relative to at least one symptom or benchmark of cancer.

“Improvement in next day functioning” or “wherein there is improvement in next day functioning” refers to improvement after waking from an overnight sleep period wherein the beneficial effect of administration of a compound according to Formula (I), or a pharmaceutically acceptable salt thereof, applies to at least one symptom of a syndrome, disease or disorder herein and is discernable, either subjectively by a subject or objectively by an observer, for a period of time, e.g., 2 hours, 3 hours, 4 hours, 5 hours, 6 hours, 12 hours, 24 hours, etc. after waking.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml or μg·hr/ml). The area under the curve is governed by clearance. Clearance is defined as the volume of blood or plasma that is totally cleared of its content of drug per unit time (ml/min).

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount”, previously referred to, can also mean a dosage sufficient to alleviate one or more symptoms of a syndrome, disorder, disease, or condition being treated, or to otherwise provide a desired pharmacological and/or physiologic effect. “Effective amount” or “therapeutically effective amount” may be used interchangeably herein.

“Co-administered with”, “administered in combination with”, “a combination of”, “in combination with” or “administered along with” may be used interchangeably and mean that two or more agents are administered in the course of therapy. The agents may be administered together at the same time or separately in spaced apart intervals. The agents may be administered in a single dosage form or in separate dosage forms.

“Subject in need thereof” includes individuals that have been diagnosed with cancer. The methods may be provided to any individual including, e.g., wherein the subject is a neonate, infant, a pediatric subject (6 months to 12 years), an adolescent subject (age 12-18 years) or an adult (over 18 years). Subjects include mammals. “Subject” and “patient” are used interchangeably herein.

“Analog” and “Derivative” may be used interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

### EXAMPLES

The examples provided herein are included solely for augmenting the disclosure herein and should not be considered to be limiting in any respect.

### Example 1

Quantitative Comparison of Compound A with Other BCRP Inhibitors. To illustrate Compound A's superior inhibition efficiency, the table below compares its IC₅₀ value with well-established BCRP inhibitors.

| Inhibitor | IC₅₀ (μM) | Selectivity | Toxicity Concerns |
|---|---|---|---|
| Compound A | 0.0065 | High | Low |
| Ko143 | 0.11 | Moderate | Low |
| Fumitremorgin C | ~1.0 | High | High (neurotoxicity) |
| Gefitinib | 0.3 | Low | Moderate |
| Imatinib Mesylate | 0.17 | Low | Moderate |
| Ritonavir | 19.5 | Low | High |
| Novobiocin | 0.05-0.1 | Moderate | Low |

The table demonstrates Compound A superior potency compared to Ko143 and FTC, while also minimizing toxicity concerns.

### Example 2

Mechanism of Action and Transporter Interaction. Beyond its potent inhibition of BCRP, Compound A was evaluated for interactions with other key transporters:
P-gp Inhibition: Partial inhibition (<25%) at 1.5 μM.
MATE1 Inhibition: IC₅₀ of 6.8 μM, indicating weak inhibition
MATE2-K Inhibition: Partial inhibition (<40%) at 4.5 μM
OATP1B1 and OATP1B3 Inhibition: Minimal inhibition observed

These findings confirm Compound A as a highly selective BCRP inhibitor with limited interference with other transporters, reducing the likelihood of drug-drug interactions.

### Example 3

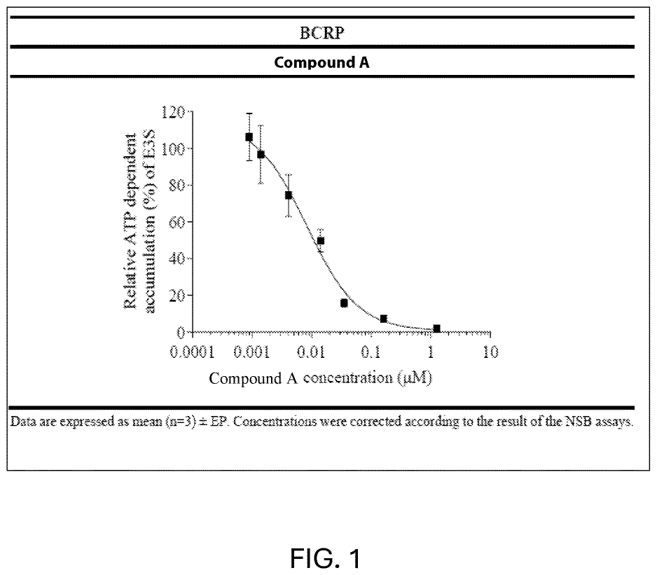

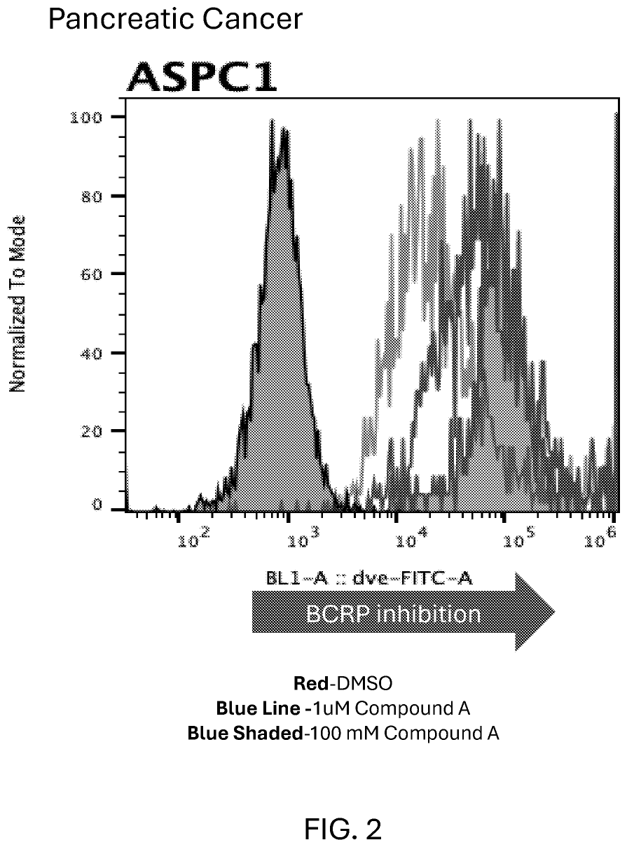

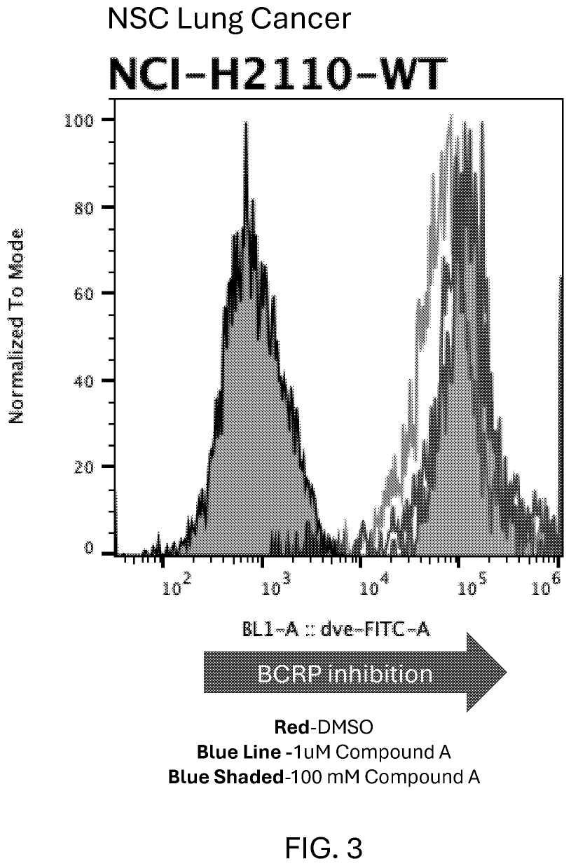

Compound A potently inhibited BCRP in a lipid vesicle assay (IC50=8 nM). See, FIG. 1. Data are expressed as mean (n=3)+/−EP. Concentrations were corrected according to the results of non-specific binding (NSB) assays. Compound A inhibited transporter efflux including BCRP in cultured cell lines AsPC-1 and NCI-H2110-wt. See, FIGS. 2 and 3.

AsPC-1 is a well-known human pancreatic cancer cell line used in cancer research, derived from a 62-year-old female patient's ascites fluid. It is a common model for studying pancreatic adenocarcinoma and is used to test the effectiveness of various treatments, including chemotherapy and novel therapies. NCI-H2110-wt is a human lung cancer cell line which is used to study non-small cell lung cancer (NSCLC), specifically the wild-type (wt) version. The cell line originates from a patient with NSCLC and is a standard tool for drug screening and biological assays.

BCRP (ABCG2) is an ATP-binding cassette efflux transporter that uses ATP to transport substrates such as estrone-3-sulfate (E3S) across membranes. Vesicles prepared from AsPC-1 plasma membranes expose the nucleotide-binding site and substrate-binding site to the medium, enabling direct access of ATP, E3S, and Compound A. In the presence of ATP+Mg²⁺, BCRP accumulates [³H]-E3S inside vesicles (ATP condition); in AMP or no-ATP, uptake is low and defines background. Decrease ATP-dependent [³H]-E3S uptake in a concentration-dependent manner; IC₅₀ is obtained from the inhibition curve. See, FIG. 1.

### Example 4

A Cell Line Derived Xenograft HT29 xenograft mouse model (CDx HT29 mice) (female Balb/c nude), average age 5-9 weeks and greater than 17 gm at inoculation was used to determine the effect of administration of Compound A on trastuzumab deruxtecan (DS-8201) inhibition of tumor growth in the CDx HT29 mouse model. HT-29 is a human colon cancer cell line that was used to create the CDX (Cell Line Derived Xenograft) HT29 xenograft mouse model. The HT29 xenograft model enables analysis of the anti-tumor efficacy of anticancer drugs and may be used to elucidate the mechanism of acquired resistance to tyrosine kinase inhibitors (TKIs).

DS8201 is used to treat certain types of cancer, including HER2-positive metastatic breast, gastric, and non-small cell lung cancers. DS-8201 is an antibody-drug conjugate composed of an anti-HER2 (human epidermal growth factor receptor 2) antibody, a cleavable tetrapeptide-based linker, and a cytotoxic topoisomerase I inhibitor. The antibody delivers topoisomerase I to cancer cells having the HER2 protein where topoisomerase I exerts its chemotherapeutic effect, thus killing the cancer cells.

### Study Design:

### Cell Line Derived Xenograft HT29 Xenograft Mouse Model:

The HT-29 cancer cells were maintained in vitro with McCoy's 5a medium supplemented with 10% fetal bovine serum at 37 °C in an atmosphere of 5% CO₂ in the air. The cells in exponential growth phase were harvested and quantitated by cell counter before tumor inoculation. Each mouse was inoculated subcutaneously in the right front flank region with HT-29 tumor cells (3×10⁶) in 0.1 ml of PBS for tumor development. The randomization was started when the mean tumor size reached approximately 121 mm³. A total of 80 mice were enrolled in the study and allocated into 10 groups, with 8 mice per group. Randomization was performed based on “Matched distribution” method. The date of grouping was denoted as day 0. The treatment was initiated on the same day (day 0) as randomization per study design.

After tumor inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), squinting/orbital tightening and any other abnormalities. Mortality and observed clinical signs were recorded for individual animals in detail. Tumor volumes were measured twice per week after randomization in two dimensions using a caliper, and the volume was expressed in mm³ using the formula: V=(L×W×W)/2, where V was tumor volume, L is tumor length (the longest tumor dimension) and W was tumor width (the longest tumor dimension perpendicular to L). Dosing as well as tumor and body weight measurements were conducted in a Laminar Flow Cabinet.

### Statistical Analysis:

### Comparisons Between Two Groups,

1. Shapiro-Wilk Normality test was run to test the normality. (At least 3 data points in a group).
2. If one or more p-values of Shapiro-Wilk tests were less than or equal to 0.05, then it indicated that the data did not follow normal distribution, Mann-Whitney U test was run to test the significant difference between the medians of the two groups. If p-value was greater than 0.05, Welch's t-test was run to test the significant difference between the means of the two groups.
   Comparisons Among More than Two Groups,
1. Bartlett's test was run to test homogeneity of variance.
2. If p-value of Bartlett's test was greater than 0.05, the null hypothesis of homogeneity of variance could not be rejected. ANOVA was run to test whether there existed any pair of groups with different group means, and the post hoc Tukey HSD was run for all pairwise comparisons or Dunnett's t test was run for each treatment against one control.
3. If p-value of Bartlett's test was less than or equal to 0.05, Kruskal-Wallis test was run to test the group medians' difference in any pair of groups, and the post hoc Conover's non-parametric all-pairs comparison test was run for all pairwise comparisons, or Conover's non-parametric many-to-one comparison test was run for each treatment against one control. Both of the tests used single-step p-value adjustment.

In addition, the pairwise comparisons without multiple comparison correction and reported nominal/uncorrected p-values were performed directly by Welch's t-test or Mann-Whitney U test. The criterion of the Welch's t-test or Mann-Whitney U test was the same as the two-group comparison method mentioned above.

### Tolerability Phase:

The tolerability of a daily dose of 5 mg/kg (IP) Compound A in 6.25% DMSO/93.75% Captisol (proprietary name for sulfobutylether-β-cyclodextrin, or SBE-β-CD) (50% g/mL Captisol in purified water) in water buffer at a dose volume of 10 ml/kg for 5 days was assessed in 3 female tumor bearing mice.

### Efficacy Assessment Phase:

Dosing with DS8201 was conducted for 23 days with dosing once weekly (QW).

DS8201 was dosed by itself (IV) in phosphate buffer solution (PBS) at 1, 3, and 10 mg/kg QW×4 doses.

In the Compound A weekly co-dosing combination groups OV369 IP in vehicle (6.25% DMSO/93.75% v/v (50% w/v Captisol in purified water) (0.05 mg/mL) was co-dosed intraperitoneally (at a fixed dose of 5 mg/kg (IP) with the DS8201 dosed intravenously (IV) in PBS at doses of 1, 3, and 10 mg/kg at a frequency of once a week for 23 days to assess if Compound A would potentiate the efficacy of DS8201 by inhibition of BCRP which would prevent efflux of the payload from cancer cells.

In the Compound A daily co-dosing combination groups Compound A in vehicle (6.25% DMSO/93.75% v/v (50% w/v Captisol in purified water) (0.05 mg/mL) was co-dosed daily at a fixed dose of 5 mg/kg (IP) for 23 days along with the weekly dosing of DS8201.

Compound A in vehicle (6.25% DMSO/93.75% v/v (50% w/v Captisol in purified water) was also dosed by itself daily for 23 days at a dose of 5 mg/kg (IP).

The study involved three cohorts.

Cohort 1: DS8201 intravenously (IV) weekly, 1 mg/kg, 3 mg/kg and 10 mg.

Cohort 2: DS8201 IV weekly+Compound A intraperitoneally (IP) weekly; 3 mg/kg DS8201 weekly+5 mg/kg Compound A IP weekly; 10 mg/kg DS8201 weekly+5 mg/kg Compound A IP weekly.

Cohort 3 DS8201 (dosed weekly) and Compound A: 5 mg/kg IP daily; 1 mg/kg IP daily; 3 mg/kg IP daily; 10 mg/kg IP daily.

Doses were instituted when tumor volume (TV)=100-150 mm³. Cohorts 1, 2 and 3 received the respective first doses on day 1 of the study. Cohorts 1 and 2 received respective doses on day 8, day 16, day 21 and day 22. Cohort 3 received daily doses from day 1 through day 22. During initial tumor growth (Day 4), Compound A (dosed daily or weekly) potentiated up to 3× the tumor inhibition at all doses of DS8201. In later stages (≥day 7), potentiation by Compound A was less pronounced but generally persistent.

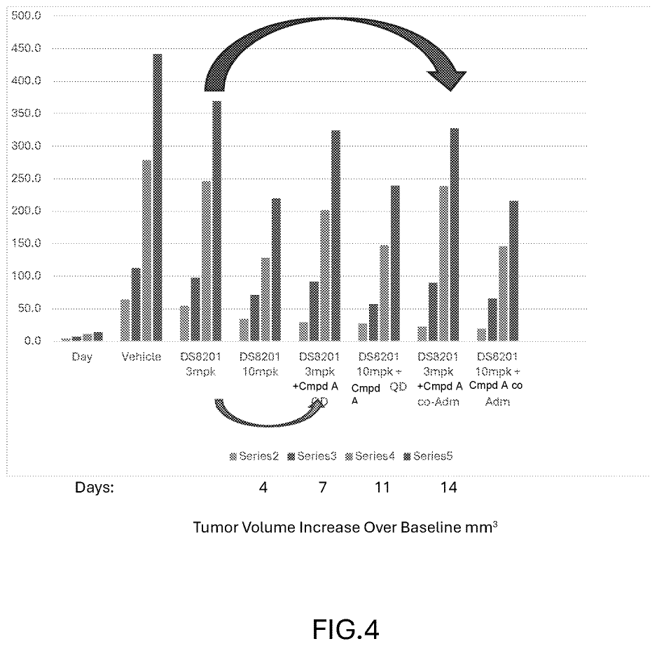

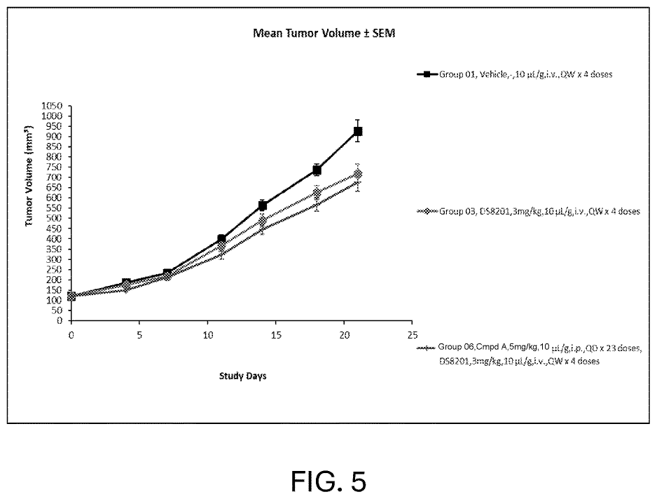

As can be seen from FIG. 4 and FIG. 5 both weekly and daily (QD) dosing of Compound A potentiates the effect of DS8201 inhibition of tumor growth in the CDx mouse model. Vehicle (6.25% DMSO/93.75% Captisol (proprietary name for sulfobutylether-β-cyclodextrin, or SBE-3-CD) in water (50% g/mL Captisol in purified water): allowed continuous tumor growth. DS8201: 10 mg/kg more effective (slower tumor growth) vs 3 mg/kg. 5 mg/kg IP Compound A daily: potentiates DS82101 at 3 mg/kg more than at 10 mg/kg. Potentiation by Compound A more robust on Day 4 of early stage small, growing tumor.

At 5 mg/kg Compound A IP, Compound A plasma levels in mice at 0.3 h=148 ng/ml=37 nM free (5×IC50 of BCRP). In a single ascending dose (SAD) phase in healthy volunteers, at 50 mg IV, 0.3 h Compound A plasma concentration=249 ng/ml.

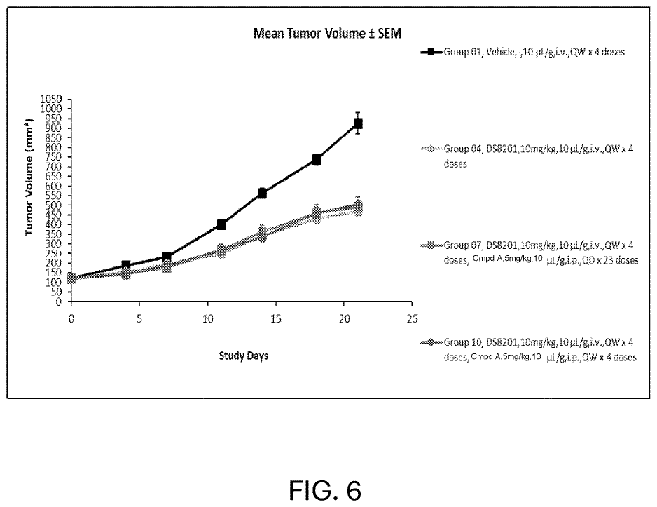

In sum, Compound A was well tolerated, with no mortality or body weight loss observed in animals that received Compound A. Compound A showed a trend to enhance the anti-tumor efficacy of DS-8201 in the HT-29 model. This was more obvious in the early phase of the dosing (especially Day 4 and 7 in week one). DS-8201 showed a dose-related increase in Dxd (cytotoxic warhead of DS-8201) levels in both plasma and tumor samples. Compound A caused an increase in tumor levels of Dxd at DS-8201 dose levels of 1 mg/kg and 3 mg/kg (daily dosing of Compound A), but not at 10 mg/kg. See, FIG. 6. The likely mechanism of action is the inhibition of BCRP, which prevents the efflux of Dxd from tumor cells, thereby enhancing the tumor inhibition efficacy of DS-8201. Overall, there was a trend indicating that Compound A enhances the anti-tumor efficacy of DS-8201 correlating with Compound A mediated increases in tumor levels of Dxd resulting from Compound A mediated inhibition of BCRP which effluxes DxD from tumor cells.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method for treating cancer comprising administering a composition comprising a compound according to Formula (I), 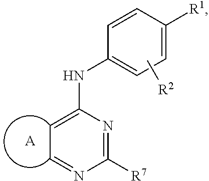
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 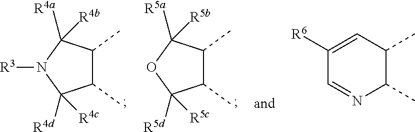
or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and -C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to a subject in need thereof.

2. The method for treating cancer according to claim 1, wherein about 0.001 mg to about 1500 mg of the compound according to Formula (I), or a pharmaceutically acceptable salt thereof, is administered to the subject.

3. The method for treating cancer according to claim 1, wherein about 0.01 mg/kg to about 500 mg/kg of the compound according to Formula (I), or a pharmaceutically acceptable salt thereof, is administered to the subject.

4. The method for treating cancer according to claim 1, wherein the compound according to Formula (I), or a pharmaceutically acceptable salt thereof, is Compound A:

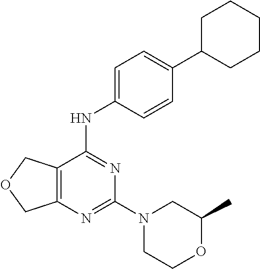 [SMILES: C[C@@H]1CN(c2nc3c(c(Nc4ccc(C5CCCCC5)cc4)n2)COC3)CCO1]

5. The method for treating cancer according to claim 1, wherein the compound according to Formula (I), or a pharmaceutically acceptable salt thereof, is Compound B:

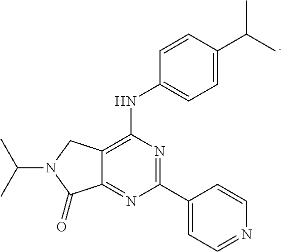 [SMILES: CC(C)c1ccc(Nc2nc(-c3ccncc3)nc3c2CN(C(C)C)C3=O)cc1]

6. The method for treating cancer according to claim 1, wherein the compound according to Formula (I), or a pharmaceutically acceptable salt thereof, is Compound C:

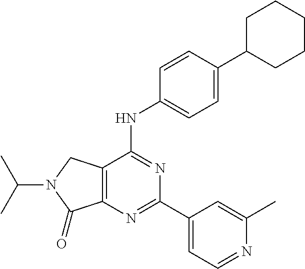 [SMILES: Cc1cc(-c2nc(Nc3ccc(C4CCCCC4)cc3)c3c(n2)C(=O)N(C(C)C)C3)ccn1]

7. The method for treating cancer according to claim 1, wherein the compound according to Formula (I), or a pharmaceutically acceptable salt thereof, is Compound D:

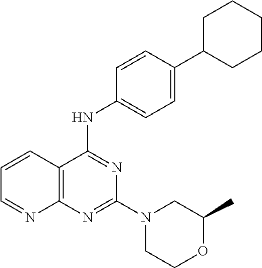 [SMILES: C[C@@H]1CN(c2nc(Nc3ccc(C4CCCCC4)cc3)c3cccnc3n2)CCO1]

8. The method for treating cancer according to claim 1, wherein the compound according to Formula (I), or a pharmaceutically acceptable salt thereof, is Compound E:

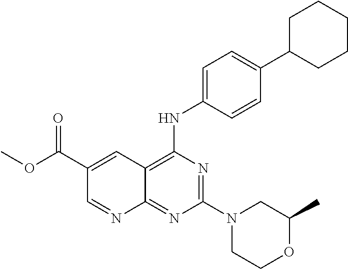 [SMILES: COC(=O)c1cnc2nc(N3CCO[C@H](C)C3)nc(Nc3ccc(C4CCCCC4)cc3)c2c1]

9. The method for treating cancer according to claim 1, wherein the compound according to Formula (I), or a pharmaceutically acceptable salt thereof, is Compound F:

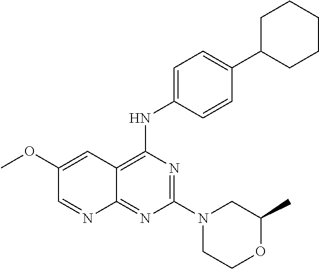 [SMILES: COc1cnc2nc(N3CCO[C@H](C)C3)nc(Nc3ccc(C4CCCCC4)cc3)c2c1]

10. The method for treating cancer according to claim 1, wherein the compound according to Formula (I), or a pharmaceutically acceptable salt thereof, is Compound G:

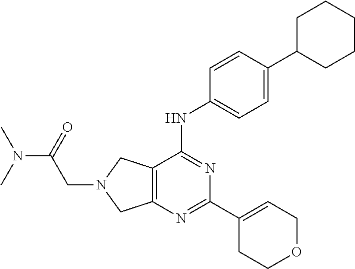 [SMILES: CN(C)C(=O)CN1Cc2nc(C3=CCOCC3)nc(Nc3ccc(C4CCCCC4)cc3)c2C1]

11. The method for treating cancer according to claim 1, wherein the compound according to Formula (I), or a pharmaceutically acceptable salt thereof, is Compound H:

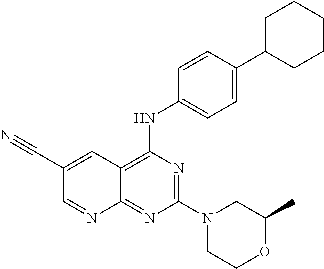 [SMILES: C[C@@H]1CN(c2nc(Nc3ccc(C4CCCCC4)cc3)c3cc(C#N)cnc3n2)CCO1]

12. The method for treating cancer according to claim 1, wherein the cancer expresses breast cancer resistance protein (BCRP).

13. The method for treating cancer according to claim 12, wherein activity of the breast cancer resistance protein (BCRP) is inhibited by the administration.

14. The method for treating cancer according to claim 1, wherein the cancer is medulloblastoma, glioma, breast cancer, squamous cell cancer, melanoma, lung cancer, small cell lung cancer, cancer of the peritoneum, hepatocellular cancer, gastric cancer, colon cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, leptomeningeal carcinomatosis, or chronic myeloblastic leukemia or subtypes thereof.

15. The method for treating cancer according to claim 1, wherein the subject is human.

16. The method for treating cancer according to claim 1, wherein the subject is also undergoing one or more cancer therapies selected from the group consisting of surgery, chemotherapy, radiotherapy, photodynamic therapy, thermotherapy, immunotherapy, hormone therapy, and laser therapy.

17. The method for treating cancer according to claim 1, wherein the compound according to Formula (I), or a pharmaceutically acceptable salt thereof, is administered in combination with one or more chemotherapeutic agents selected from the group consisting of abraxane, actinomycin, afatinib, alitretinoin, all-trans retinoic acid, altretamine, aminocamptothecin, amuvatinib, azacitidine, azathioprine, belotecan, bendamustine, bexarotene, bisatrene, bleomycin, bortezomib, busulfan, cabazitaxel, camptothecin, carboplatin, carboquone, carmustine, capecitabine, cisplatin, chlorambucil, chlormethine, chlorozotocin, cladribine, clofarabine, cyclophosphamide, cytarabine, dacarbazine, daunorubicin, decitabine, diflomotecan, docetaxel, doxifluridine, doxorubicin, epirubicin, epothilone, erlotinib, estrone-3-sulfate (E3 S), etoposide, exatecan, flavpiridol, fludarabine, fluorouracil, fotemustine, gefitinib, gemcitabine, gimatecan, hydroxyurea, idarubicin, ifosfamide, imatinib, irinotecan, ixabepilone, lapatinib, larotaxel, lomustine, melphalan, melphalan flufenamide, mercaptopurine, methotrexate, mitobronitol, mitomycin c, mitoxantrone, nelarabine, nimustine, nitrosoureas, oxaliplatin, paclitaxel, pemetrexed, pipobroman, ranimustine, rhodamine 123, romidepsin, rosuvastatin, semustine, SN-38, streptozotocin, sulfasalazine, tafluposide, taxotere, temozolomide, tesetaxel, teniposide, teniposide 3, 9-aminocamptothecin, thiotepa, tioguanine, topotecan, treosulfan, tretinoin, triaziquone, triethylenemelamine, valrubicin, vemurafenib, vinblastine, vincristine, vindesine, vinorelbine, vismodegib, vorinostat, zidovudine, ABT-869, AMG-706, AMN-107, iodine-131, lutetium-177, lutetium-177 dotatate, actinium-225 psma, radium-223, yttrium-90 microspheres, ⁹⁰Y-Ibritumomab, tiuxetan, ¹³¹I-Tositumomab, aminolevulinic acid, silicon phthalocyanine Pc 4, m-tetrahydroxyphenylchlorin, mono-L-aspartyl chlorin e6 (NPe6), porfimer sodium, verteporfin, temoporfin, methyl aminolevulinate, hexylaminolevulinate, pheophorbide A, protoporphyrin IX, benzoporphyrin derivatives, talaporfin sodium, motexafin lutetium, HPPH, sulfonated aluminium phthalocyanines, rostaporfin, fimaporfin, azadipyrromethenes, amuvatinib, AST-487, axitinib (AG-013736), AZD-152HQPA, AZD-2171, BIBF-1120, BIRB-796, BMS-540215, bosutinib, cabozantinib, canertinib (Cl-1 033), CHIR-258/TKI-258, crizotinib, dasatinib, DMBI, dovitinib, everolimus, EXEL-2880/GSK-1363089, GW-786034, JNJ-28312141, Ki-20227, Ki8751, masitinib (AB-1 01 0), midostaurin (PKC-412), motesanib, neratinib (HKI-272), nilotinib, OSI-930, pazopanib, PD-173955, PLX-4720, ponatinib, PTK-787, quizartinib (AC220), R406, regorafenib, SKI-606, sorafenib, staurosporine, SU-14813, sunitinib, tandutinib (MLN-518), telatinib, temsirolimus, tivozanib, vandetanib, and vatalanib.

18. The method for treating cancer according to claim 1, wherein the compound according to Formula (I), or a pharmaceutically acceptable salt thereof, is administered enterally.

19. The method for treating cancer according to claim 1, wherein the compound according to Formula (I), or a pharmaceutically acceptable salt thereof, is administered parenterally.

20. The method for treating cancer according to claim 1, wherein the compound according to Formula (I), or a pharmaceutically acceptable salt thereof, is administered in the form of a liquid for parenteral use, a tablet, a capsule, a caplet, a pill, an oral liquid, a lozenge, a film, a powder, an aerosol, or a patch.

21. A method for treating a cancer that expresses breast cancer resistance protein (BCRP) comprising delivering a compound according to Formula (I), 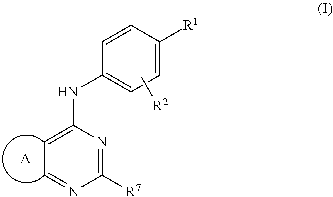
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 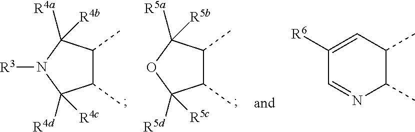
or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to the cancer.

22. A method for treating a condition in a subject wherein treatment with a therapeutic agent is inhibited by BCRP activity, the method comprising administering to the subject an effective amount of a compound according to Formula (I), 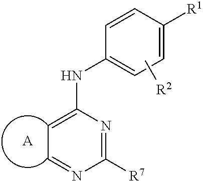
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 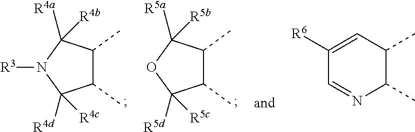
or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
in combination with a therapeutic agent useful for treating the condition, wherein the compound according to Formula (I), or a pharmaceutically acceptable salt thereof, increases the concentration of the therapeutic agent in a target tissue or cell relative to administration of the therapeutic agent alone.

23. A method for inhibiting activity of breast cancer resistance protein (BCRP) in cancer comprising administering an effective amount of a compound according to Formula (I), 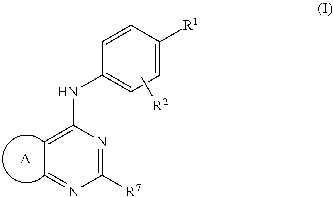
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 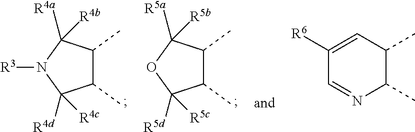
or a N-oxide thereof;
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to the cancer.

24. A composition comprising a compound according to Formula (I), 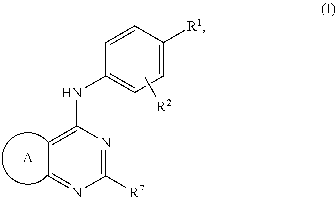
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 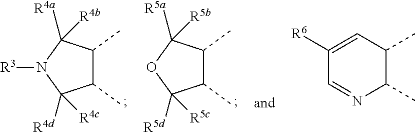
or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
and a chemotherapeutic agent selected from the group consisting of abraxane, actinomycin, afatinib, alitretinoin, all-trans retinoic acid, altretamine, aminocamptothecin, amuvatinib, azacitidine, azathioprine, belotecan, bendamustine, bexarotene, bisatrene, bleomycin, bortezomib, busulfan, cabazitaxel, camptothecin, carboplatin, carboquone, carmustine, capecitabine, cisplatin, chlorambucil, chlormethine, chlorozotocin, cladribine, clofarabine, cyclophosphamide, cytarabine, dacarbazine, daunorubicin, decitabine, diflomotecan, docetaxel, doxifluridine, doxorubicin, epirubicin, epothilone, erlotinib, estrone-3-sulfate (E3 S), etoposide, exatecan, flavpiridol, fludarabine, fluorouracil, fotemustine, gefitinib, gemcitabine, gimatecan, hydroxyurea, idarubicin, ifosfamide, imatinib, irinotecan, ixabepilone, lapatinib, larotaxel, lomustine, melphalan, melphalan flufenamide, mercaptopurine, methotrexate, mitobronitol, mitomycin c, mitoxantrone, nelarabine, nimustine, nitrosoureas, oxaliplatin, paclitaxel, pemetrexed, pipobroman, ranimustine, rhodamine 123, romidepsin, rosuvastatin, semustine, SN-38, streptozotocin, sulfasalazine, tafluposide, taxotere, temozolomide, tesetaxel, teniposide, teniposide 3, 9-aminocamptothecin, thiotepa, tioguanine, topotecan, treosulfan, tretinoin, triaziquone, triethylenemelamine, valrubicin, vemurafenib, vinblastine, vincristine, vindesine, vinorelbine, vismodegib, vorinostat, zidovudine, ABT-869, AMG-706, AMN-107, iodine-131, lutetium-177, lutetium-177 dotatate, actinium-225 psma, radium-223, yttrium-90 microspheres, ⁹⁰Y-Ibritumomab, tiuxetan, ¹³¹I-Tositumomab, aminolevulinic acid, silicon phthalocyanine Pc 4, m-tetrahydroxyphenylchlorin, mono-L-aspartyl chlorin e6 (NPe6), porfimer sodium, verteporfin, temoporfin, methyl aminolevulinate, hexylaminolevulinate, pheophorbide A, protoporphyrin IX, benzoporphyrin derivatives, talaporfin sodium, motexafin lutetium, HPPH, sulfonated aluminium phthalocyanines, rostaporfin, fimaporfin, azadipyrromethenes, amuvatinib, AST-487, axitinib (AG-013736), AZD-152HQPA, AZD-2171, BIBF-1120, BIRB-796, BMS-540215, bosutinib, cabozantinib, canertinib (Cl-1 033), CHIR-258/TKI-258, crizotinib, dasatinib, DMBI, dovitinib, everolimus, EXEL-2880/GSK-1363089, GW-786034, JNJ-28312141, Ki-20227, Ki8751, masitinib (AB-1 01 0), midostaurin (PKC-412), motesanib, neratinib (HKI-272), nilotinib, OSI-930, pazopanib, PD-173955, PLX-4720, ponatinib, PTK-787, quizartinib (AC220), R406, regorafenib, SKI-606, sorafenib, staurosporine, SU-14813, sunitinib, tandutinib (MLN-518), telatinib, temsirolimus, tivozanib, vandetanib, and vatalanib.

25. The composition according to claim 24, wherein the composition is included in a formulation suitable for enteral administration or a formulation suitable for parenteral administration.

26. A method for decreasing activity of breast cancer resistance protein (BCRP) comprising contacting cells expressing BCRP with a compound according to Formula (I), 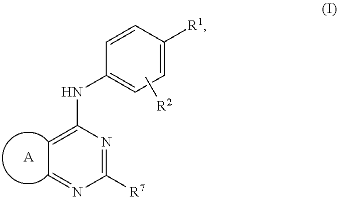
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 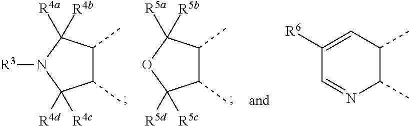
or a N-oxide thereof,
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
or a pharmaceutically acceptable salt thereof;
wherein said contacting causes the activity of BCRP in the cells to decrease.

27. The method for decreasing activity of breast cancer resistance protein (BCRP) according to claim 26, wherein said contacting is accomplished by administration the compound according to Formula (I), or a pharmaceutically acceptable salt thereof, in vivo.

28. The method for decreasing activity of breast cancer resistance protein (BCRP) according to claim 26, wherein said contacting is accomplished by administration the compound according to Formula (I), or a pharmaceutically acceptable salt thereof, in vitro.

29. A method for reducing multidrug resistance (MDR) in cancer chemotherapy comprising administering an effective amount of a compound according to Formula (I), 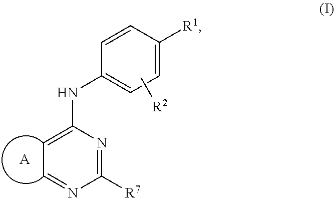
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 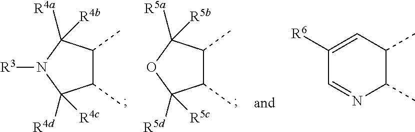
or a N-oxide thereof;
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
to a subject in need thereof.

30. The method for reducing multidrug resistance (MDR) in cancer chemotherapy according to claim 29, wherein about 0.001 mg to about 1500 mg of the compound according to Formula (I), or a pharmaceutically acceptable salt thereof, is administered to the subject.

31. The method for reducing multidrug resistance (MDR) in cancer chemotherapy according to claim 29, wherein about 0.01 mg/kg to about 500 mg/kg of the compound according to Formula (I), or a pharmaceutically acceptable salt thereof, is administered to the subject.

32. The method for reducing multidrug resistance (MDR) in cancer chemotherapy according to claim 29, wherein the cancer is medulloblastoma, glioma, breast cancer, squamous cell cancer, melanoma, lung cancer, small cell lung cancer, cancer of the peritoneum, hepatocellular cancer, gastric cancer, colon cancer, pancreatic cancer, glioblastoma, cervical cancer, ovarian cancer, testicular cancer, bladder cancer, colorectal cancer, endometrial or uterine carcinoma, salivary gland carcinoma, kidney or renal cancer, prostate cancer, vulval cancer, thyroid cancer, head and neck cancer, B-cell lymphoma, chronic lymphocytic leukemia, acute lymphoblastic leukemia, hairy cell leukemia, leptomeningeal carcinomatosis, or chronic myeloblastic leukemia or subtypes thereof.

33. The method for reducing multidrug resistance (MDR) in cancer chemotherapy according to claim 29, wherein the subject is a mammal.

34. The method for reducing multidrug resistance (MDR) in cancer chemotherapy according to claim 33, wherein the mammal is human.

35. The method for reducing multidrug resistance (MDR) in cancer chemotherapy according to claim 29, wherein the subject is undergoing one or more cancer therapies selected from the group consisting of surgery, chemotherapy, radiotherapy, photodynamic therapy, thermotherapy, immunotherapy, hormone therapy, and laser therapy.

36. The method for reducing multidrug resistance (MDR) in cancer chemotherapy according to claim 29, wherein the compound according to Formula (I), or a pharmaceutically acceptable salt thereof, is administered in combination with one or more chemotherapeutic agents selected from the group consisting of abraxane, actinomycin, afatinib, alitretinoin, all-trans retinoic acid, altretamine, aminocamptothecin, amuvatinib, azacitidine, azathioprine, belotecan, bendamustine, bexarotene, bisatrene, bleomycin, bortezomib, busulfan, cabazitaxel, camptothecin, carboplatin, carboquone, carmustine, capecitabine, cisplatin, chlorambucil, chlormethine, chlorozotocin, cladribine, clofarabine, cyclophosphamide, cytarabine, dacarbazine, daunorubicin, decitabine, diflomotecan, docetaxel, doxifluridine, doxorubicin, epirubicin, epothilone, erlotinib, estrone-3-sulfate (E3 S), etoposide, exatecan, flavpiridol, fludarabine, fluorouracil, fotemustine, gefitinib, gemcitabine, gimatecan, hydroxyurea, idarubicin, ifosfamide, imatinib, irinotecan, ixabepilone, lapatinib, larotaxel, lomustine, melphalan, melphalan flufenamide, mercaptopurine, methotrexate, mitobronitol, mitomycin c, mitoxantrone, nelarabine, nimustine, nitrosoureas, oxaliplatin, paclitaxel, pemetrexed, pipobroman, ranimustine, rhodamine 123, romidepsin, rosuvastatin, semustine, SN-38, streptozotocin, sulfasalazine, tafluposide, taxotere, temozolomide, tesetaxel, teniposide, teniposide 3, 9-aminocamptothecin, thiotepa, tioguanine, topotecan, treosulfan, tretinoin, triaziquone, triethylenemelamine, valrubicin, vemurafenib, vinblastine, vincristine, vindesine, vinorelbine, vismodegib, vorinostat, zidovudine, ABT-869, AMG-706, AMN-107, iodine-131, lutetium-177, lutetium-177 dotatate, actinium-225 psma, radium-223, yttrium-90 microspheres, ⁹⁰Y-Ibritumomab, tiuxetan, ¹³¹I-Tositumomab, aminolevulinic acid, silicon phthalocyanine Pc 4, m-tetrahydroxyphenylchlorin, mono-L-aspartyl chlorin e6 (NPe6), porfimer sodium, verteporfin, temoporfin, methyl aminolevulinate, hexylaminolevulinate, pheophorbide A, protoporphyrin IX, benzoporphyrin derivatives, talaporfin sodium, motexafin lutetium, HPPH, sulfonated aluminium phthalocyanines, rostaporfin, fimaporfin, azadipyrromethenes, amuvatinib, AST-487, axitinib (AG-013736), AZD-152HQPA, AZD-2171, BIBF-1120, BIRB-796, BMS-540215, bosutinib, cabozantinib, canertinib (Cl-1 033), CHIR-258/TKI-258, crizotinib, dasatinib, DMBI, dovitinib, everolimus, EXEL-2880/GSK-1363089, GW-786034, JNJ-28312141, Ki-20227, Ki8751, masitinib (AB-1 01 0), midostaurin (PKC-412), motesanib, neratinib (HKI-272), nilotinib, OSI-930, pazopanib, PD-173955, PLX-4720, ponatinib, PTK-787, quizartinib (AC220), R406, regorafenib, SKI-606, sorafenib, staurosporine, SU-14813, sunitinib, tandutinib (MLN-518), telatinib, temsirolimus, tivozanib, vandetanib, and vatalanib.

37. The method for reducing multidrug resistance (MDR) in cancer chemotherapy according to claim 29, wherein the compound according to Formula (I), or a pharmaceutically acceptable salt thereof, is administered enterally.

38. The method for reducing multidrug resistance (MDR) in cancer chemotherapy according to claim 29, wherein the compound according to Formula (I), or a pharmaceutically acceptable salt thereof, is administered parenterally.
